(19) United States
(12) Patent Application Publication
(10) Pub. No.: US 2015/0335763 A1
(43) Pub. Date: Nov. 26, 2015

(54) HMGB1-BINDING BEADS AND USES THEREOF

(71) Applicant: THE FEINSTEIN INSTITUTE FOR MEDICAL RESEARCH, Manhasset, NY (US)

(72) Inventors: Sangeeta Chavan, Syosset, NY (US); Jacob Levine, Fresh Meadows, NY (US); Yehuda Tamari, Oyster Bay, NY (US); Kevin J. Tracey, Old Greenwich, CT (US); Huan Yang, Oakland Gardens, NY (US)

(73) Assignee: The Feinstein Institute For Medical Research, Manhasset, NY (US)

(21) Appl. No.: 14/759,681

(22) PCT Filed: Jan. 9, 2014
(86) PCT No.: PCT/US14/10818

§ 371 Date: Jul. 8, 2015

Related U.S. Application Data
(60) Provisional application No. 61/750,440, filed on Jan. 9, 2013.

(51) Int. Cl. A61K 47/48; A61K 31/7125; A61K 31/711; A61K 9/00; A61K 9/50

(52) U.S. Cl. CPC A61K 47/48853; A61K 9/0053; A61K 9/0065; A61K 9/5005; A61K 31/711; A61K 31/7125

(57) ABSTRACT

Disclosed are therapeutic beads comprising agents, such as nucleic acids, that bind to high mobility group box 1 (HMGB1) and methods of treating subjects with conditions that would benefit from reducing the deleterious effects of HMGB1, such as inflammatory bowel diseases, comprising administering the beads to the gastrointestinal tract of the subjects.

### CROSS-REFERENCE TO RELATED APPLICATION

The application claim the benefit of U.S. Provisional Patent Application No. 61/750,440, filed Jan. 9, 2013, the contents of which are herein incorporated by reference in their entirety.

### FIELD OF THE INVENTION

The invention relates to beads comprising agents that bind to high mobility group box 1 (HMGB1) and methods of treating subjects with an inflammatory cascade, an inflammatory bowel disease, Crohn's disease, colitis, ulcerative colitis, colitis-associated cancer or any condition that would benefit from reducing the deleterious effects of HMGB1, comprising administering the beads to the gastrointestinal tract of the subjects.

### BACKGROUND OF THE INVENTION

Throughout this application various publications are referred to in parentheses. Full citations for these references may be found at the end of the specification before the claims. The disclosures of these publications are hereby incorporated by reference in their entireties into the subject application to more fully describe the art to which the subject application pertains.

Inflammatory bowel diseases (IBDs) are chronic remitting and relapsing disorders of the gastrointestinal (GI) tract with unknown etiology and without specific therapy. The contributing factors for IBDs are genetic, environmental and immunological defects. There are two major types of inflammatory bowel diseases, ulcerative colitis and Crohn's disease. Unlike Crohn's disease, which can affect any part of the gastrointestinal tract, ulcerative colitis characteristically impairs the mucosal lining of the colon and rectum. Current treatment involves attempts to block the inflammatory activation by using local and systemic anti-inflammatory or immunomodulatory agents (18).

IBD is one of the five most prevalent gastrointestinal diseases in the USA, with an overall cost of more than $1.7 billion. As many as 1.4 million individuals in the USA and 2.2 million individuals in Europe suffer from IBD. In the United States, about one million people are affected with ulcerative colitis (1), and the annual medical costs are in the billion dollar range. In the USA, IBD accounts for more than 700,000 physician visits, 100,000 hospitalizations, and disables 119,000 patients annually. Additional therapeutic approaches are clearly needed.

HMGB1 has been implicated in infection and in sterile inflammation. More recently, HMGB1 has been shown to be involved in the development of murine colitis and colitis-associated cancer (2). HMGB1 is abundantly found in stools of IBD patients and is a novel bio-marker of intestinal inflammation and in the diagnosis of pediatric IBD (4). Inhibiting HMGB1 release by ethyl pyruvate is able to ameliorate colitis and reduces intestinal cytokine production in IL-10 knockout mice (3).

HMGB1 binds DNA with certain sequences or certain structures (6-8, 19-20). Some of this DNA has been used to bind and remove HMGB1 in the treatment of inflammatory diseases, such as in endotoxemia and experimental autoimmune encephalomyelitis (6-8). However, direct injection of DNA to animals might cause side effects, such as generation of auto-antibodies or other deleterious conditions. The present invention addresses the need for treatment of conditions in which it is desirable to remove HMGB1, such as inflammatory bowel diseases, using procedures that do not lead to the undesirable side effects associated with direct injection of DNA.

### SUMMARY OF THE INVENTION

The invention provides therapeutic beads for treating an inflammatory cascade and inflammatory bowel diseases such as Crohn's disease, colitis, ulcerative colitis, colitis-associated cancer or a condition that would benefit from reducing the deleterious effects of HMGB1, comprising beads coated with agents that bind to HMGB1.

The invention also provides methods for treating subjects with an inflammatory cascade and/or inflammatory bowel diseases such as Crohn's disease, colitis, ulcerative colitis, colitis-associated cancer or a condition that would benefit from reducing the deleterious effects of HMGB1, the methods comprising administering beads coated with agents that bind to HMGB1 to the gastrointestinal tract of a subject in an amount effective to treat the disease or condition.

The invention also provides methods of reducing the level of HMGB1 in the gastrointestinal tract of a subject comprising administering beads coated with agents that bind to HMGB1 to the gastrointestinal tract of the subject in an amount effective to reduce the level of HMGB1 in the gastrointestinal tract of a subject.

### BRIEF DESCRIPTION OF THE DRAWINGS

FIG. 1A. Beads with respective oligos. A diagram showing four different DNA-coated bead types with their respective oligo sequences, type and structure of oligo sequences, and the affinity for HMGB1. SEQ ID NO:1 and SEQ ID NO:2 are linear DNA oligos. Four way junction DNA are composed of 4 oligos complementary to each. Duplex DNA was generated using 2 DNA oligos.

FIG. 1B. Strategy to link oligo to beads. Figure shows a strategy to link DNA oligonucleotides to beads. The immobilization of DNA oligos to CNBr-activated sepharose beads with carboxyl linker was performed according to manufacturer's instructions (see Methods).

FIG. 1C. Ethidium bromide staining of beads to confirm conjugation of DNA to beads. Figure demonstrates the conjugation of DNA oligonucleotides to beads. To confirm the conjugation of DNA oligos to sepharose beads, beads (before and after DNA conjugation) were stained with ethidium bromide (1 μg/ml) for 30 min at room temperature. After washing, beads were exposed to ultraviolet light. The positive staining of beads indicates the presence of DNA on all four DNA-beads after conjugation. Data are representative of 7 separate experiments.

FIG. 2A. Depletion of HMGB1 by beads. Figure shows a depletion of HMGB1 in the incubation supenatant by the incubation of DNA oligonucleotide-coated beads B1, B2 and B3 but not B4, and corresponding increase in the amount of HMGB1 bound to the beads. Depletion curve of HMGB1 binding to various DNA beads. Recombinant HMGB1 (2 μg per reaction) was incubated with increasing amounts of different types of DNA-beads as indicated at 4° C. overnight. The mixture was then centrifuged at 2,000 rpm for 5 minutes to precipitate the beads. Supernatants were collected and HMGB1 content was measured by Western blot analysis. Beads were washed with PBS for 5 times, boiled and eluates were subjected to Western blot for HMGB1.

FIG. 2B. Depletion curve. Figure represents the data in FIG. 2A as a set of curves. Data are from 3-5 experiments.

FIG. 3A. Binding capacity of the beads. Figure shows the binding capacity of the beads. Beads B1, B2 and B3 sequestered HMGB1 from the incubation solution in a concentration dependent manner while empty control beads captured insignificant amounts of HMGB1. Fixed amount (20 μl drained beads) of DNA-beads containing SEQ ID NO:1, SEQ ID NO:2, duplex or 4 way junction DNA was incubated with increasing amounts HMGB1 (50 μl) at concentrations indicated for 2 hours at room temperature with rotation. The mixture was then centrifuged at 2,000 rpm for 5 minutes to precipitate the beads. The supernatants were aspirated, both supernatants and eluate of beads were subjected to Western blot for HMGB1 measurement using monoclonal anti-HMGB1 antibodies. Binding of 1 μg HMGB1 requires about 0.4 ng (SEQ ID NO:1 or SEQ ID NO:2 DNA) or 2.8 ng (4 way junction DNA) in beads, respectively.

FIG. 3B. Saturation curve. Figure represents the data in FIG. 3A as a set of saturation curves. Data are from 3 experiments.

FIG. 4A. Time course of HMGB1 binding to beads. Figure shows time course of HMGB1 binding to B1, B2 and B3 beads. Various beads containing SEQ ID NO:1, SEQ ID NO:2 or 4 way junction DNA (5 μl) were incubated with 500 ng of HMGB1 in PBS (50 μl total volume) and incubated at room temperature for the time periods indicated. HMGB1 bound to the beads was revealed by Western blot analysis. The data shows that B3 binds much faster to HMGB1 than B1 or B3.

FIG. 4B. Time course of HMGB1 binding to beads. Figure represents the data in FIG. 4A as a set of curves. Data are from 2 experiments.

FIG. 5A. Oligos are inert. SEQ ID NO:1, SEQ ID NO:2 and 4 way junction DNA are inert. Murine macrophage-like RAW 264.7 cells were incubated with HMGB1 (positive control) or SEQ ID NO:1, SEQ ID NO:2 or 4 way junction oligos as indicated for 16 hours. TNF released in the supernatants was measured by commercially obtained ELISA kits. Data are mean+SEM from 3 experiments. *: p<0.05 vs. HMGB1 alone.

FIG. 5B. S1, S2 and S3 neutralize HMGB1 inflammatory activity. Figure shows that SEQ ID NO:1, SEQ ID NO:2 and 4 way junction DNA inhibit HMGB1-induced TNF release from macrophages. HMGB1 induced TNF release by macrophages is reduced by the presence of S1, S2 and S3 in concentration dependent manner.

FIG. 5C. Beads do not induce cell death. Figure shows that the beads are not toxic. Incubation of cells with increasing concentrations and time does not cause increased cell death.

FIG. 6. Beads bind to different forms of HMGB1. Figure shows that different forms of HMGB1 can be captured by beads B1 comprising SEQ ID NO:1. Increasing amounts of HMGB1 (100, 250 and 500 ng) were added to SEQ ID NO:1 beads (20 μl) and the mixture (50 μl total volume) was incubated at room temperature for 2 hours. The mixture was then centrifuged and HMGB1 bound to beads were revealed by Western blotting with anti-HMGB1 antibodies. N=1 experiment.

FIG. 7A-7D. Binding capacity of DNA beads to HMGB1 in the presence of acid, heparin and plasma. Beads (2, 5 and 10 μl) containing SEQ ID NO:1, SEQ ID NO:2 or 4 way junction DNA were incubated with 500 ng of HMGB1 in the presence or absence of (A) 2 or 10 U/ml of heparin at room temperature for 2 hours, or (B) 20 μl cow's plasma for 1 hour at room temperature, or (C) 20 μl of cow's plasma and heparin (10 U/ml) for 1 hour at room temperature, or (D) HCl (pH 1 or 2) for 1 hour at room temperature. After washing beads with PBS 5 times, HMGB1 in the beads was revealed by Western blot analysis using anti-HMGB1 antibodies. Data are representative from 3 experiments.

FIG. 8A. Beads capture HMGB1 from cell supernatant. Figure shows that B1 and B2 beads capture HMGB1 from supernatants of activated cells. RAW 264.7 cells in 6-well plate were stimulated with LPS (100 ng/ml) for 16 hours, and HMGB1 containing supernatant was collected and concentrated 10 times through centrifugation with Microcon centrifugal filters. The RAW264.7 cell supernatant was then incubated with beads containing control, beads comprising SEQ ID NO:1 or SEQ ID NO:2 at room temperature for 1 hour with rotation. HMGB1 content in both supernatant and beads was measured by Western blot. N=2 repeats each performed in duplicate.

FIG. 8B. Beads capture HMGB1 from sepsis serum. DNA beads remove HMGB1 from septic mice sera. Serum (20 μl) from normal or septic mice was incubated with 50 μl of SEQ ID NO:2-containing or control beads at room temperature for 1 hour. Samples were then centrifuged at room temperature for 5 minutes. Binding of DNA-beads with HMGB1 was evaluated by using Western blot or ELISA kit. Data shown are means+SEM from 3-5 mice per group. *: P<0.05 vs. untreated septic serum.

FIG. 9A. Disease severity in mice with DSS-colitis. Figure shows that mice with DSS-colitis lose body weight, have increased levels of HMGB1 and TNF in serum and colons respectively, and have significantly shorter and heavier colons. Female BALB/c mice (8-12 weeks old, n=4-5 per group) were fed with 2% dextran sodium sulfate (DSS, weight/volume) dissolved in drinking water ad libitum for 5 days, and then switched to normal water for 2 days. Control mice received the same water without DSS. Mice were weighed daily, and monitored for the presence of gross blood in feces. Mice were euthanized on day 8 after overnight fasting. Full length colons and blood were collected for analyses. Body weight changes over time (FIG. 9A, left), colon length and weight, serum HMGB1 and TNF released from colon culture on day 8 after DSS treatment (see methods, FIG. 9A, right) were measured. *: P<0.05 vs. control.

FIG. 9B. Beads capture HMGB1 from colitis colon. Figure shows B1 and B2 beads but not empty beads capture HMGB1 from colitis colons. The full length colons were isolated from colitis and control mice (Methods). Colon was isolated and tied at both ends to avoid leaking, and infused with 0.5 ml of 50% beads slurry containing SEQ ID NO:1 or SEQ ID NO:2 DNA. The preparation was incubated at room temperature for 2 hours with gentle shaking. The beads were then recovered from the colon and washed with PBS 3-5 times to remove non-specific binding. Binding of HMGB1 from intestinal segments of DSS-induced colitis mice ex vivo was then analyzed by Western blot probed with anti-HMGB1 antibodies. Data shown are representative of 3-4 mice per group.

FIG. 9C. Beads capture HMGB1 from colitis feces. Figure shows that B1 and B2 beads, but not the empty beads, bound and removed HMGB1 from the fecal samples obtained from colitis mice. Stools in the colon were gently flushed out with cold PBS, and the suspension was rotated overnight at 4° C. in the presence of gentamycin and imipenem. After centrifugation to remove fecal debris, the supernatant that contains protein was incubated with beads containing SEQ ID NO:1 or SEQ ID NO:2 at room temperature for 2 hours with rotation. At the end of incubation, beads were recovered, washed extensively with PBS, and eluates from beads were subjected to Western blot probed with anti-HMGB1 antibodies. Data shown are representative of 3-4 mice per group.

FIG. 10A. Administration of neutralizing anti-HMGB1 antibody improves body weight in colitis mice. Figure shows that neutralization of HMGB1 by administrering anti-HMGB1 antibody improves body weight in DSS-colitis mice. Female BALB/c mice (20 mice per group) were given 4% DSS in drinking water for 5 days to induce colitis, and then switched to normal water for three days. Mice received intraperitoneal injection of monoclonal anti-HMGB1 antibodies or control IgG at 10 μg/mouse on days 0, 1, 2, 4 and 6 after DSS administration and were euthanized on day 8^{th}. Treatment with anti-HMGB1 antibody increased body weight in DSS-induced colitis mice. *: p<0.05 vs. control IgG group. N=20/group.

FIG. 10B. Administration of neutralizing anti-HMGB1 antibody reduces colon weight and fecal HMGB1 levels. Figure shows that administration of anti-HMGB1 antibody improves colon weight and reduces fecal HMGB1 levels. Colon and serum measurements of DSS-induced mice treated with anti-HMGB1 antibodies. Colon measurements (length and weight), serum and fecal HMGB1 levels in colitis mice treated with anti-HMGB1 antibody or control IgG. *: p<0.05 vs. IgG group. N=20 mice per group.

FIG. 10C. Administration of neutralizing anti-HMGB1 antibody reduces tissue injury in colons. Figure shows that DSS-colitis mice have significantly reduced inflammatory infiltrate and colonic wall thickening when treated with anti-HMGB1 antibodies. Histological evaluation of HMGB1 antibody or IgG-treated colitis mice. Representative histology of colon H&E staining is shown from normal, DSS plus treatment with HMGB1 antibody or IgG at 8^{th} day after initiation of DSS administration. *:P<0.05 vs. IgG group. N=20 mice. Magnification: ×40.

FIG. 11A. Administration of B2 beads improves body weight in colitis mice. Figure shows that administering B2 beads to DSS-colitis mice ameliorates body weight loss. Female BALB/c mice (10 mice per group) were given 4% DSS in drinking water to induce colitis water for 5 days to induce colitis, and then switched to normal water. Mice were orally administrated with 300 μl (50% slurry, gavage) of B2 or empty beads on days 0, 2, 4 and 6 after DSS initiation and were euthanized on day 8^{th}. Treatment with B2 beads increased body weight in DSS-induced colitis mice. *: p<0.05 vs. empty beads group.

FIG. 11B. Administration of B2 beads reduces colon weight and fecal HMGB1 levels. Figure shows that administration of B2 beads improves colon weight and reduces fecal and serum HMGB1 levels in DSS-colitis mice. Colon measurements (weight and length) and levels of serum and fecal HMGB1 in colitis mice treated with B2 or empty beads. *: p<0.05 vs. empty beads group.

FIG. 11C. Administration of B2 beads reduces tissue injury in colons. Figure shows that DSS-colitis mice have significantly reduced inflammatory infiltrate and colonic wall thickening when treated with B2 beads. Histological evaluation of B2 or empty beads treated colitis mice. Representative H&E staining of colons from normal, empty or B2 beads-treated colitis mice is shown. Histological scores (see Methods) of colons are shown. N=10 mice per group. *: p<0.05 vs. empty beads group. Magnification: ×40.

FIG. 12A. Administration of B2 beads to IL10 knock-out mice with colitis improves body weight. Figure shows that administering B2 beads to IL10 knock-out mice that develop spontaneous colitis improves body weight. Twelve weeks old female IL-10 KO mice were orally administrated with 300 μl (50% slurry, gavage) of B2 or empty beads three times a week for a total of six weeks. Treatment with B2 beads increased body weight in mice. *: p<0.05 vs. empty beads group. N=5-7 mice per group.

FIG. 12B. Administration of B2 beads to IL10 knock-out mice with colitis reduces colon weight and cytokines and serum HMGB1 levels. Figure shows that administration of B2 beads to IL10 knock out mice improves colon weight, and reduces serum HMGB1 levels and colonic IL1 and IL6 levels. Colon and serum measurements of IL-10 KO mice treated with B2 or empty beads. Colon measurements (weight, expression of IL-6 and IL-1B mRNA) and serum HMGB1 levels in IL-10 KO mice treated with B2 or empty beads. *: p<0.05 vs. empty beads group. N=5-7 mice per group.

FIG. 12C. Administration of B2 beads to IL10 knock-out mice with colitis reduces tissue injury in colons. Figure shows that when IL10 knock out mice have significantly reduced colonic wall thickening when treated with B2 beads. Histological evaluation of B2 or empty beads-treated IL-10 KO mice. Representative H&E staining of colon from wild type (C57) or IL-10 KO mice and histological scores (see Methods) of colons are shown. *: P<0.05 vs. empty beads group. N=5-7 mice per group. Magnification: ×40.

### DETAILED DESCRIPTION OF THE INVENTION

The invention provides a method of treating a subject with a disease or condition selected from the group consisting of an inflammatory cascade or inflammatory bowel disease, Crohn's disease, colitis, ulcerative colitis, colitis-associated cancer and a condition that would benefit from reducing the deleterious effects of HMGB1, the method comprising administering beads coated with one or more agents that bind to HMGB1 in the gastrointestinal tract of the subject in an amount effective to treat the disease or condition. Reducing the deleterious effects of HMGB1 can be accomplished, for example, by depleting the levels of HMGB1 or reducing the activity of HMGB1.

The invention also provides a method of reducing the level of HMGB1 in the gastrointestinal tract of a subject comprising administering beads coated with one or more agents that bind to HMGB1 in the gastrointestinal tract of the subject in an amount effective to reduce the level of HMGB1 in the gastrointestinal tract of a subject.

The invention also provides therapeutic beads for treating an inflammatory cascade or inflammatory bowel disease, Crohn's disease, colitis, ulcerative colitis, colitis-associated cancer or a condition that would benefit from reducing the deleterious effects of HMGB1 comprising beads coated with one or more agents that bind to HMGB1. The agents that coat the beads may be attached to the beads covalently or non-covalently.

The beads can be in a composition formulated, for example, for administration through the mouth or for rectal administration.

The beads can be administered, for example, through the mouth. The beads can be administered, for example, using a tube or an endoscope, such that, for example, the beads can be administered to the small intestine and/or large intestine but not to the stomach. The beads can be administered rectally, for example by retention enema.

The beads can be, for example, sepharose beads or polystyrene latex beads. Polystyrene microspheres (diameter 1.00-1.99 μm) can be obtained, for example, from Bangs Laboratories, Inc.

The beads can have an enteric coating. The coating can protect an agent such as a nucleic acid from damage due to, for example, acidic contents of the stomach. The coating can dissolve in the alkaline environment of the small intestine. Materials that can be used for enteric coatings include, for example, fatty acids, waxes, shellac, plastics, and/or plant fibers.

Preferably, the beads not absorbable by the gastrointestinal tract.

Administration of the beads to a subject can reduce the level of HMGB1 in the subject's gastrointestinal tract and stool.

The beads can have a diameter of, e.g., 1 to 1,000 microns, e.g. 1 to 100 microns or 1-2 microns.

The agent that binds HMGB1 can comprise, for example, one or more of an antibody, an antibody fragment, a peptide, a small synthetic compound, a peptide nucleic acid and/or a nucleic acid that binds HMGB1. The antibody can be a monoclonal antibody or a polyclonal antibody. The antibody fragment can be, e.g., a F(ab′)₂ fragment or a Fab′ fragment. The synthetic compound can have a molecular weight of, e.g., 2,000 daltons or less, e.g., 1,000-2,000 daltons. A peptide nucleic acid has a backbone composed of repeating N-(2-aminoethyl)-glycine units linked by peptide bonds.

Preferably, the agent comprises a nucleic acid. Preferably, the nucleic acid is or comprises DNA. The nucleic acid can be, for example, a single linear chain, a duplex of two chains, a 4-way junction of four chains, a cisplatin-modified nucleic acid, a kinked nucleic acid, a hemi-catenated nucleic acid, or a nucleic acid containing a loop.

A preferred nucleic acid is a single linear chain consisting of 15-30 nucleotides, e.g. a nucleic acid consisting of 20 nucleotides. The nucleic acid can comprise a sequence that is at least 80% or 90% identical to the sequence X1GX2ATGAGX3TTCCTGATGCT (SEQ ID NO:9), where X1 and X2 are independently A, C, G or T, and X3 is C or G. The nucleic acid can comprise the sequence X1GX2ATGAGX3TTCCTGATGCT (SEQ ID NO:9), where X1 and X2 are independently A, C, G or T, and X3 is C or G. The nucleic acid can comprise a sequence that is at least 80% or 90% identical to the sequence AGCATGAGGTTCCTGATGCT (SEQ ID NO:1). The nucleic acid can comprise the sequence AGCATGAGGTTCCTGATGCT (SEQ ID NO:1). The nucleic acid can comprise a sequence that is at least 80% or 90% identical to the sequence TGGATGAGCTTCCTGATGCT (SEQ ID NO:2). The nucleic acid can comprise the sequence TGGATGAGCTTCCTGATGCT (SEQ ID NO:2).

The nucleic acid can consist essentially of the sequence X1GX2ATGAGX3TTCCTGATGCT (SEQ ID NO:9), where X1 and X2 are independently A, C, G or T, and X3 is C or G. The nucleic acid can consist essentially of the sequence AGCATGAGGTTCCTGATGCT (SEQ ID NO:1). The nucleic acid can consist essentially of the sequence TGGATGAGCTTCCTGATGCT (SEQ ID NO:2). A used herein, a nucleic acid consists essentially of the sequence in SEQ ID NO:1, SEQ ID NO:2 or SEQ ID NO:9 if the additions to SEQ ID NO:1, SEQ ID NO:2 or SEQ ID NO:9 do not diminish the ability of the nucleic acid to bind HMGB1 compared, respectively, to the ability of SEQ ID NO:1, SEQ ID NO:2 or SEQ ID NO:9 to bind HMGB1.

| Human HMGB1 is reported to have the amino acid sequence | |
|---|---|
| Accession CAG33144, Version CAG33144.1, GI:48145843 | |
| (SEQ ID NO: 10) | |
| 1 | mgkgdpkkpr gkmssyaffv qtcreehkkk hpdasvnfse fskkcserwk tmsakekgkf |
| 61 | edmakadkar yeremktyip pkgetkkkfk dpnapkrpps afflfcseyr pkikgehpgl |
| 121 | sigdvakklg emwnntaadd kqpyekkaak lkekyekdia ayrakgkpda akkgvvkaek |
| 181 | skkkkeeeed eedeedeeee edeededeee ddddd |
| or | |
| Accession CAE48262, Version CAE48262.1 GI:37515993. | |
| (SEQ ID NO: 11) | |
| 1 | mgkgdpkkpr gkmssyaffv qtcreehkkk hpdasvnfse fskkcserwk tmsakekgkf |
| 61 | edmakadkar yeremktyip pkgetkkkfk dpnapkrpps afflfcseyr pkikgehpgl |
| 121 | sigdvakklg emwnntaadd kqpyekkaak lkekyekdia ayrakgkpda akkgvvkaek |
| 181 | skkkkeeeed eedeedeeee edeededeee dddde |

The nucleic acid can consist of the sequence X1GX2ATGAGX3TTCCTGATGCT (SEQ ID NO:9), where X1 and X2 are independently A, C, G or T, and X3 is C or G. The nucleic acid can consist of the sequence AGCATGAGGTTCCTGATGCT (SEQ ID NO:1). The nucleic acid can consist of the sequence TGGATGAGCTTCCTGATGCT (SEQ ID NO:2).

The nucleic acid can comprise a 4-way junction of four chains where each chain comprises a sequence that is at least 80% or 90% identical to the sequence set forth in SEQ ID NO:5, SEQ ID NO:6, SEQ ID NO:7 or SEQ ID NO:8. The nucleic acid can comprise a 4-way junction of four chains where each chain comprises the sequence set forth in SEQ ID NO:5, SEQ ID NO:6, SEQ ID NO:7, or SEQ ID NO:8.

The nucleic acid can comprise a duplex of two chains where each chain comprises a sequence that is at least 80% or 90% identical to the sequence set forth in SEQ ID NO:3 or SEQ ID NO:4. The nucleic acid can comprise a duplex of two chains where each chain comprises the sequence set forth in SEQ ID NO:3 or SEQ ID NO:4.

The nucleic acid can have, for example, a phosphorothioate backbone or a phosphodiester backbone. Preferably, for nucleic acids comprising SEQ ID NO:1, SEQ ID NO:2 or SEQ ID NO:9, the nucleic acid has a phosphorothioate backbone.

The nucleic acid can have a backbone between nucleotide bases of, for example, —O—P(═O)S—O—, where O— is the point of attachment to a base.

The agent can be attached to the beads by, for example, a covalent bond or a non-covalent bond. The nucleic acid can be attached to the beads using, for example, a carbon amino linker, which can comprise, for example, NH2(CH2)6O—PO2-O-DNA, where DNA represents the nucleic acid.

Preferably, the agent is non-immunogenic and does not induce cellular toxicity.

The beads can be administered to the subject acutely, chronically, or episodically, as required.

This invention will be better understood from the Experimental Details that follow. However, one skilled in the art will readily appreciate that the specific methods and results discussed are merely illustrative of the invention as described more fully in the claims that follow thereafter.

### EXPERIMENTAL DETAILS

### Introduction

The present studies show that DNA conjugated to beads is able to bind and remove HMGB1 efficiently in vitro, ex vivo and in vivo. Exemplary DNA-conjugated sepharose beads bound HMGB1 in a concentration-dependent manner and captured HMGB1 from RAW 264.7 cell supernatant stimulated with LPS and from mouse CLP serum. In a dextran sulfate sodium (DSS) induced-colitis model, mice had body weight loss, bloody diarrhea, shortened colon length and increased colon weight. DNA-conjugated beads captured HMGB1 during colon culture ex vivo and removed HMGB1 from stools isolated from DSS colitis mice. These data demonstrate the therapeutic potential for DNA-conjugated beads in the removal of HMGB1 in conditions where it is desirable to remove HMGB1, such as inflammatory bowel disease.

### Materials and Methods

Materials. Dextran sulfate sodium (DSS, MW=36-50 kDa) was purchased from MP Biomedicals (Solon, Ohio). CNBr-activated sepharose 4 fast flow resin was from GE Healthcare (Piscataway, N.J.). Ethidium bromide was from Bio-Rad (Hercules, Calif.). Lipopolysaccharide (LPS, E. Coli. 0111:B4) and heparin sulfate were purchased from Sigma (St. Louis, Mo.). Fetal bovine serum was obtained from Gibco BRL (Carlsbad, Calif.). Isopropyl-D-thiogalactopyranoside (IPTG) was purchased from Pierce (Rockford, Ill.).

Cell culture. RAW 264.7 cells (American type culture collection, ATCC, Rockville, Md.) were cultured in DMEM supplemented with 10% fetal bovine serum, 100 U/ml penicillin and 100 μg/ml streptomycin. Cells were used at 90% confluence and treatment was carried out in serum-free Opti-MEM I medium (Life Technologies, Carlsbad, Calif.). Human primary monocytes were purified by density gradient centrifugation through Ficoll from blood donated by healthy volunteers to the Long Island Blood Bank (New York Blood Center, Melville, N.Y.).

Generation of DNA-beads—Oligonucleotide synthesis. All oligonucleotides (SEQ ID NOs: 1-8, Table 1) were custom-made by Genemed Synthesis, Inc. (San Antonio, Tex.). An amino group linker has been conjugated to the 5′ end of the following oligos: SEQ ID NOs: 1, 2, 3 and 5. In order to prevent DNase degradation, all oligos were synthesized with phosphorothioate (for SEQ ID NOs: 1 and 2) or phosphodiester backbone (for 4 way junction and duplex) throughout the sequences.

Generation of duplex and 4-way junction DNA. The duplex DNA was generated by annealing SEQ ID NO:3 and SEQ ID NO: 4 at 70° C. for 5 min, and then slowly cooled down to room temperature. SEQ ID NOs:5-8 were mixed in equal molar ratio and heated to 70° C. for 5 min, then cooled to 25° C. over two hours through a thermocycler to generate 4 way junction DNA (7, 11).

Preparation of DNA-beads. The immobilization of DNA to beads was performed according to published literature (9) with minor modifications. Briefly, 0.5 g of the CNBr-activated sepharose 4 resin was swelled in 10 ml of cold 1 mM HCl for 15 min at room temperature. The resin was then washed by 10 ml of cold 1 mM HCl. 10 nmol of DNA was diluted in 3 ml of coupling buffer (0.1 M Boric acid, pH 8), and 20 μl of diluted oligo was saved as ‘before coupling’. DNA in coupling buffer was then applied to moist resin and rotated for 4-5 hr at room temperature. To estimate the coupling efficiency, OD260 from samples before and after coupling were measured and percentage of DNA immobilized to sepharose beads after the coupling reaction was calculated. Excess reactive groups on the resin were then blocked by using end-capping buffer (0.5 M glycine, 0.1 M boric acid, pH 8.0) at 4° C. overnight. The DNA-beads were then subject to four cycles of acidic (pH4) and basic (pH8) wash. The oligo-sepharose bead preparation was re-suspended in TE (10 mM Tris-HCl, 1 mM EDTA, pH7.6) buffer until use. Control beads went through the same procedures as above with the exception of addition of DNA in the coupling reaction. The beads coated with S1, S2, S3 and S4 DNA (see Table 1) are referred as B1, B2, B3 and B4, respectively. Approximately 8 nmol of S1 and S2 DNA (75 μg), and 4 nmol of S3 and S4 DNA (180 μg) were bound to each ml of drained beads.

Binding ratio of HMGB1 to DNA beads. Each of the three DNA-beads showed maximum binding at 1 μg HMGB1 which corresponds to 38 pmols of HMGB1. The concentration of DNA in beads is approximately 2.5 μM (12.5 pmoles/5 μl) for B1 and B2 and 1.25 μM (6.23 pmoles/5 μl) for B3. Given that 38 pmoles of HMGB1 is bound to 5 μl of DNA beads, it can be calculated that the binding ratio of HMGB1 to B1 and B2 is 3:1 (38 pmoles HMGB1 per 12.5 pmoles of DNA=3) and for B3 is 6:1 (38 pmoles HMGB1 per 6.25 pmoles of DNA =6).

FAM-labeled B2 DNA. Carboxyl terminal Fam-labeled S2 conjugated to sepharose beads were made by Genemed Synthesis Inc. FAM-labeled B2 (50 μl) was incubated with feces extract of colitis mice (300 μl) for 2 hours at 37° C. After centrifugation, beads were washed five times with PBS and re-suspended as 50% slurry. The fluorescence intensity in both supernatants and beads (before and after the incubation) was measured by using a microplate spectrophotometer (Winooski, Vt.) at excitation of 494 nm.

Ethidium bromide staining of DNA beads. Conjugation of DNA to beads was measured by staining with ethidium bromide (1 μg/ml) for 30 minutes at room temperature and then washing three times with PBS. Ethidium bromide fluorescence, which indicates the presence of bound DNA, was visualized under ultraviolet light.

Binding studies of DNA beads with HMGB1—Concentration-dependent binding of HMGB1 and DNA beads. The binding ability and affinity of the DNA beads to HMGB1 was tested using depletion approach. Drained DNA beads B1, B2, B3 and B4 (fixed amount of 20 μl) were mixed with increasing amounts HMGB1 at concentrations of 0, 0.01, 0.1, 0.2, 0.5 and 1.5 μg/50 μl and incubated at room temperature for two hours with rotation. In addition, recombinant HMGB1 proteins (fixed amount of 2 μg) were mixed with DNA beads (0, 5, 10, 20, and 40 μl drained DNA beads) to bring a total volume of 100 μl. The mixtures were incubated at room temperature for two hours with rotation. Each mixture was then centrifuged at 2,000 rpm for five minutes to separate the beads. The supernatants and eluate of the beads (obtained from boiling the beads for 5 minutes at 100° C.) were probed for HMGB1 with Western blot.

HMGB1 protein preparation, neutralizing anti-HMGB1 monoclonal antibody (mAb) and removal of LPS. Recombinant HMGB1 was expressed in E. coli and purified to homogeneity as previously described (21, 22). Mutant and redox modified HMGB1 was made as previously described (23). Anti-HMGB1 mAb was generated as described previously (24). HMGB1 was extracted with triton X-114 to remove any contaminating LPS as described previously (22). The LPS content in HMGB1 was measured by the Chromogenic Limulus Amebocyte Lysate Assay (Catalog #50-647U, Lonza Inc., Walkersville, Md.). The LPS content in protein solutions was less than 10 pg/mg protein.

Cytokine measurements. TNF and IL-6 released in the cell culture supernatants were measured by commercially obtained enzyme-linked immunosorbent assay (ELISA) kits per manufacturer's instructions (R & D System Inc., Minneapolis, Minn.).

HMGB1 measurement. HMGB1 levels were measured using Western blotting method as described previously (12). Serum levels of HMGB1 were measured using ELISA method (IBL International, Hamburg, Germany).

Surface plasmon resonance analysis. Surface plasmon resonance analysis of binding of HMGB1 to DNA was conducted using the BlAcore 3000 instrument as previously described (13, BIAcore Inc, NJ). For immobilization, biotinylated DNA oligos were injected into CMS dextran sensor chip. To evaluate binding, a series of concentrations of HMGB1 0-10 μM were then passed over the sensor chip. The association of analyte and ligand was recorded respectively by surface plasmon resonance. Results were analyzed using the software BIAeval 3.2 (BIAcore Inc.).

Binding of DNA-beads with HMGB1 in vitro—Depletion and saturation studies. The binding ability of four types of DNA-beads to HMGB1 was tested using two methods. Using depletion approach, fixed amount (20 μl drained beads) of DNA-beads containing SEQ ID NO:1, SEQ ID NO:2, duplex or 4 way junction DNA were incubated with increasing amounts of HMGB1 (50 μl) at concentrations of 0, 0.01, 0.1, 0.2, 0.5 and 1.5 μg at room temperature for 2 hours with rotation. In another approach (saturation study), fixed amount (2 μg) of recombinant HMGB1 protein was mixed with increasing amounts of DNA-beads (0, 5, 10, 20, and 40 μl of drained beads) containing SEQ ID NO:1, SEQ ID NO:2, duplex or 4 way junction DNA in a total volume of 100 μl. The mixture was incubated at 4° C. overnight with gentle shaking to facilitate binding. At the end of incubation, the mixture was then centrifuged at 2,000 rpm for 5 minutes to precipitate the beads. The supernatants were aspirated, both supernatants and eluates of beads were subjected to Western blot for HMGB1 measurement using monoclonal anti-HMGB1 antibodies (13). Binding of HMGB1 to DNA in the beads is approximately 3:1 (molar ratio) for SEQ ID NOs:1 and 2, and 6:1 for 4 way junction DNA. Binding of 1 μg HMGB1 requires about 0.4 ng (SEQ ID NO:1 or 2 DNA) or 2.8 ng (4 way junction DNA) in beads, respectively.

Time course of DNA-beads and HMGB1 binding. Beads containing SEQ ID NO:1, SEQ ID NO:2 or 4 way junction DNA (5 μl) were incubated with 500 ng of HMGB1 in PBS (50 μl total volume) at room temperature for 0, 15, 30, 60, 120, 249 or 960 minutes. HMGB1 bound to the beads was revealed by Western blot analysis.

Stability study. Beads (20 μl) containing SEQ ID NOs:1 or 2 or 4 way junction DNA were incubated with 500 ng of HMGB1 in the presence or absence of 1) HCl (pH 1 or 2) for 1 hour at room temperature; or 2) 20 μl cow's plasma for 1 hour at room temperature; or 3) 2 or 10 U/ml of heparin at room temperature for 2 hours. After washing beads with PBS, HMGB1 in the supernatant and beads were subjected to Western blot analysis as described above.

Cytotoxicity of DNA beads. Epithelial cell lines HELA and human cervical cancer cell line Caco-2 in 24-well culture plates were incubated with increasing amounts and various time periods of either empty or B2 beads at 37° C. Supernatants were collected and lactate dehydrogenase (LDH) levels analyzed using LDH cytotoxicity kit. Triton X-100 (2%) was used as a positive control.

Removal of HMGB1 by DNA-beads in vitro. RAW 264.7 cells in 6-well plates were stimulated with LPS (100 ng/ml) overnight, and supernatant was collected and concentrated 10 times through centrifugation with Microcon centrifugal filters. The RAW 264.7 cell supernatant (containing HMGB1) was then incubated with beads containing control, SEQ ID NO:1, SEQ ID NO:2 or 4 way junction DNA at room temperature for 1 hour with rotation. The abilities for DNA-beads to remove HMGB1 was assessed by comparing HMGB1 levels in supernatant and in beads before and after DNA-beads treatment through Western blot using anti-HMGB1 antibodies.

Animal experiments. Female IL-10 knockout (KO) mice on C57BL/6J background (12 weeks old, stock #002251) were purchased from JAX laboratory (Bar Harbor, Me.). Female and male C57BL/6J or BALB/c (8-12 weeks old) mice were purchased from Taconic Laboratory (Germantown, N.Y.). Mice were housed in the Feinstein Institute for Medical Research Animal Facility under standard temperature and light and dark cycle. All animal procedures were approved by the IACUC of the Feinstein Institute.

Ex vivo removal of HMGB1 from septic mice induced by cecal ligation and puncture. Male C57 mice (8-12 weeks of age) were subjected to cecal ligation and puncture (CLP) procedure. In this method, a surgically-created diverticulum of the cecum is punctured, resulting in polymicrobial peritonitis, bacteremia and sepsis (12). All animals were given a normal saline solution (subcutaneously, 20 ml/kg of body weight) resuscitation, and a single dose of antibiotics (imipenem, 0.5 mg/mouse in 200 μl sterile saline injected subcutaneously, Primaxin, Merck & Co., Inc., West Point, Pa.) 30 minutes after the surgery. Mice were euthanized at 48 hours after CLP surgery through over-exposure to CO₂. Serum from normal or septic mice (20 μl) was incubated with 50 μl of DNA-containing or control beads at room temperature for 1 hour. Samples were then centrifuged at room temperature for 10 minutes to remove beads. Binding of DNA-beads with HMGB1 was evaluated by comparing HMGB1 levels in supernatant and in beads by using Western blot or ELISA kit.

Removal of HMGB1 from intestinal tissue and from stool of DSS induced colitis. Female BALB/c mice (8-12 weeks old) were used for DSS colitis. Acute colitis was induced by feeding mice with 2% dextran sodium sulfate (DSS, weight/volume) dissolved in drinking water, which was fed ad libitum for 5 days, and then switched to normal drinking water for 2 days (2-4). Control mice received the same drinking water without DSS. Mice were observed daily for body weight change, food and water consumption, and the presence of gross blood in feces. Mice were euthanized on day 8th after overnight fasting, and full length colons were collected and measured. The full length colons thus isolated were tied at both ends to avoid leaking and were infused with 0.5 ml of 50% beads slurry containing SEQ ID NO:1 or SEQ ID NO:2 DNA; the mixture was incubated at room temperature for 2 hours with gentle shaking. The beads were then recovered from the colon and washed with PBS 3-5 times to remove non-specific binding. Capturing of HMGB1 by DNA-beads was then analyzed by Western Blot as described above. Besides colon culture, stools in the colon were gently flushed out with cold PBS, and the suspension was rotated overnight at 4° C. in the presence of gentamycin and imipenem. After centrifugation to remove fecal debris, the supernatant that contains protein was incubated with beads containing SEQ ID NO:1 or SEQ ID NO:2 at room temperature for 2 hours with rotation. At the end of incubation, beads were washed extensively with PBS, and eluates from beads were subjected to Western blot probed with anti-HMGB1 antibodies.

Treatment with anti-HMGB1 antibodies in DSS-induced colitis in mice. Female BALB/c mice (n=20 per group) were given 4% DSS in drinking water to induce colitis. Mice received intraperitoneal injection of monoclonal anti-HMGB1 antibodies or control IgG at 10 μg/mouse on days 0, 1, 2, 4 and 6 after DSS administration and were euthanized on day 8^{th}. Blood, feces in the colon and colon tissues were harvested for analysis.

Treatment with DNA beads in colitis mice. Female BALB/c (10-12 weeks old) or IL-10 KO mice at 12 weeks of age (when they spontaneously develop IBD) were orally administered (gavage) 300 μl of 50% slurry of B2 or empty beads on days 0, 1, 2, 4 and 6 after DSS administration and were euthanized on day 8^{th} after DSS (for BALB/c mice) or once every other day for a total of six weeks (for IL-10 KO mice). Body weight was monitored daily (for BALB/c mice) or every other day (for IL-10 KO mice). At the time of euthanization, full length colon was collected, colon length and weight were measured. Blood, feces and colon tissues were harvested for analysis.

Histology. Colon tissues were fixed in 10% formalin and embedded in paraffin. Five μm sections were cut and stained with hematoxylin and eosin (H&E) performed by AML Laboratory (Baltimore, Md.). Colitis scores were determined for each high power view (magnification 40×) and 10 fields were viewed for each sample. The histological scoring system to quantify the degree of colitis was described previously and was evaluated in a blinded fashion. The score ranged from 0 to 14 and was defined as follows. The inflammation severity was scored as 0-3 (0, no sign of inflammation; 1, mild inflammation; 2, moderate inflammation; 3, severe inflammation). The inflammation extent was graded from 0 to 3 (0, no inflammation; 1, mucosa; 2, mucosa and submucosa; 3, transmural). Crypt damage was scored as 0 to 4 (0, no damage; 1, basal 1/3 damage; 2, basal 2/3 damage; 3, crypts loss with presence of surface epithelium; 4, loss of both crypts and surface epithelium). Percentage of involvement was defined as 0 to 4 (0, 0%; 1, 1-25%; 2, 26-50%; 3, 51-75%; 4, 76-100%). The 10 data points for each mouse were averaged and colon inflammation score was expressed as means±SEM.

Quantitative PCR analysis of colonic cytokine expression. A 0.5 cm segment from each proximal and distal end of the colon tissue was mixed together for RNA extraction using QIAzol Lysis Reagent. The levels of IL-6 and IL-1β mRNA were analyzed by quantitative PCR using a one step RT-PCR kit and a Roche Light Cycler 480 instrument. Primers sequences used in PCR amplification were as follows: IL-6 forward 5′GCTACCAAACTGGATATAATCAGGA3′ (SEQ ID NO:12) and reverse 5′CAGGTAGCTATGGTACTCCAGAA3′ (SEQ ID NO:13); IL-113 forward 5′AGTTGACGGACCCCAAAAG3′ (SEQ ID NO:14) and reverse 5′AGCTGGATGCTCTCATCAGG3′ (SEQ ID NO:15). The PCR amplification was performed by denaturing at 95° C. for 10 min, followed by 45 cycles of denaturing at 95° C. for 10 seconds, annealing at 60° C. for 30 seconds, an extension at 72° C. for 60 seconds. Relative mRNA expression was normalized to the expression of HPRT housekeeping gene (27).

Statistical analysis. Data are presented as means+/−SEM unless otherwise stated. Differences between treatment groups were determined by Student's t test, one-way ANOVA followed by the least significant difference test or regression analysis. P values less than 0.05 were considered statistically significant.

### Results and Discussion

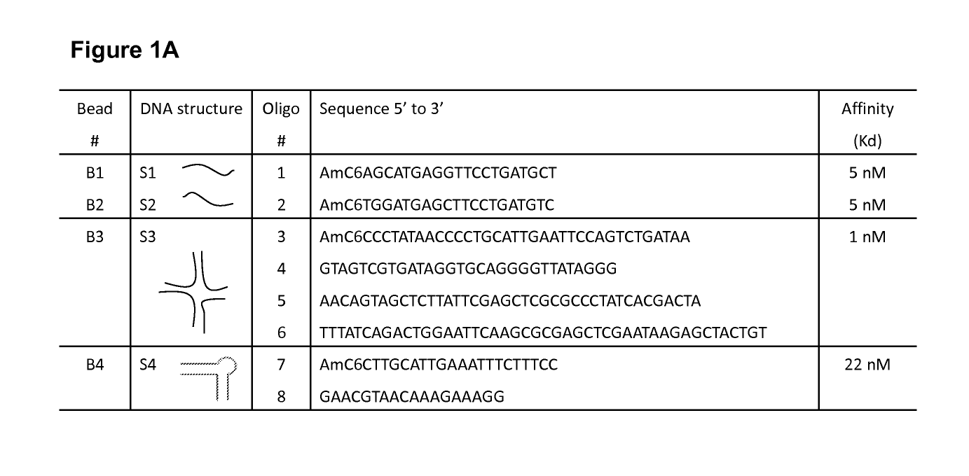

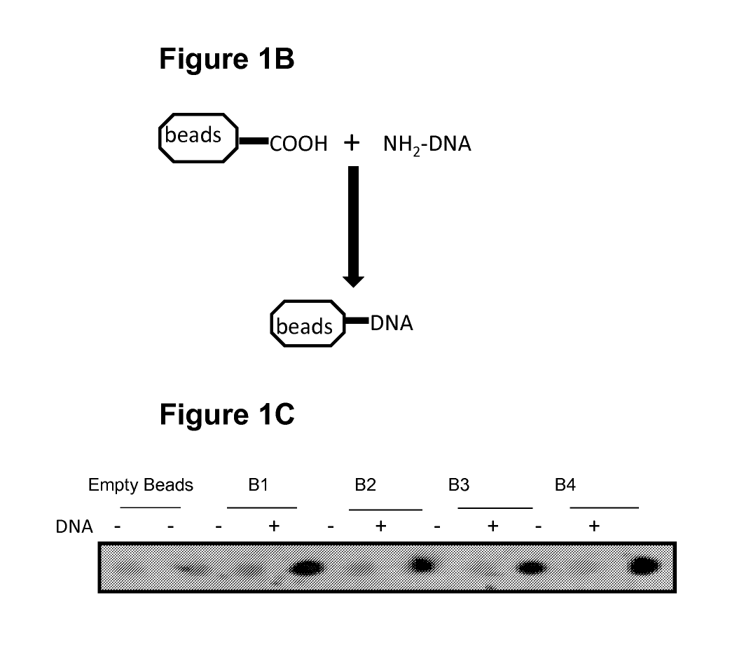

Generation of DNA conjugated beads. The present studies aimed to develop methods to remove extracellular HMGB1 from animals with conditions such as sepsis or inflammatory bowel disease by using DNA linked to beads. Since HMGB1 binds kinked DNA structures with high affinity (19), four DNA constructs were originally chosen (FIG. 1A). 1) Two similar non-immunogenic DNA oligonucleotides (oligos or ODNs) that bind HMGB1 and suppress immune response (IC 50=1 μM (8). 2) 4-way junction DNA. Hill et al. reported that 4-way junction DNA binds HMGB1 with high affinity (Kd=10-9M, 80 nM) (6, 11). 3) Kinked duplex DNA, with binding affinity to HMGB1 of 20 nM and IC50=10 nM based on cell migration assay in endothelial cells. In order to link these DNA oligos to sepharose beads and to reduce nuclease degradation, an amino linker was added at the 5′ end and oligos were synthesized on the phosphodiester backbone (FIG. 1B). To ascertain the conjugation of DNA oligos to beads (with carboxyl linker), DNA concentration was measured before and after coupling reaction and the amount of DNA immobilized to beads was calculated. The conjugation of DNA on beads was further confirmed by using ethidium bromide staining of beads (FIG. 1C). As expected, control beads, with no DNA added, had negative staining by ethidium bromide, whereas all 4 oligos had positive staining (FIG. 1C).

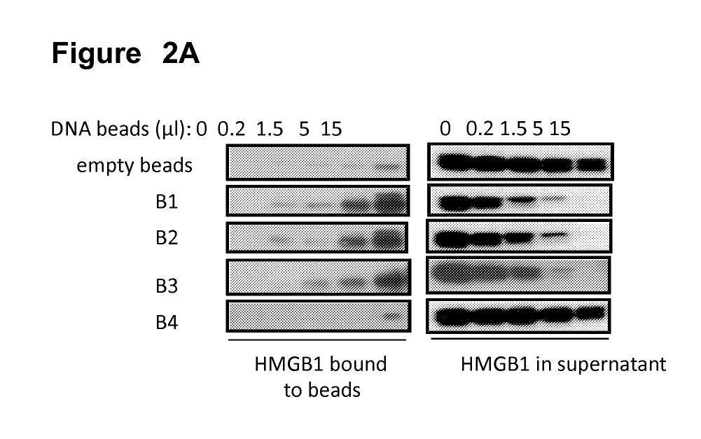

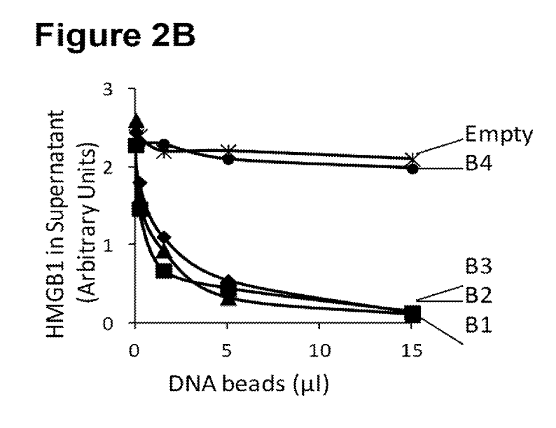

DNA-beads bind HMGB1 rapidly and with high affinity. To test the binding abilities of DNA-beads to HMGB1, recombinant HMGB1 (2 μg) was added in increasing amounts to DNA-beads. After incubating the mixture of each type of DNA bead and HMGB1, measurements were made of HMGB1 remaining in the supernatant and HMGB1 bound to the beads. As shown in FIG. 2A, compared to empty beads (without DNA), beads with SEQ ID NO:1, SEQ ID NO:2 and 4 way junction DNA bind HMGB1 efficiently (with 1 μg HMGB1 per 5 μg beads) and in a DNA concentration-dependent manner, whereas duplex DNA did not. With only 5 μl of beads (equivalent to 0.4 ng of DNA), it could bind up to 90% of the 1 μg HMGB1 added in the mixture (FIGS. 2A and 2B). In agreement with these findings, increasing amounts of HMGB1 were detected in beads confirming the binding of DNA beads to HMGB1 (FIGS. 2A and 2B). Since kinked duplex DNA beads (beads B4) did not show any binding to HMGB1 in vitro, it was eliminated for any further analyses.

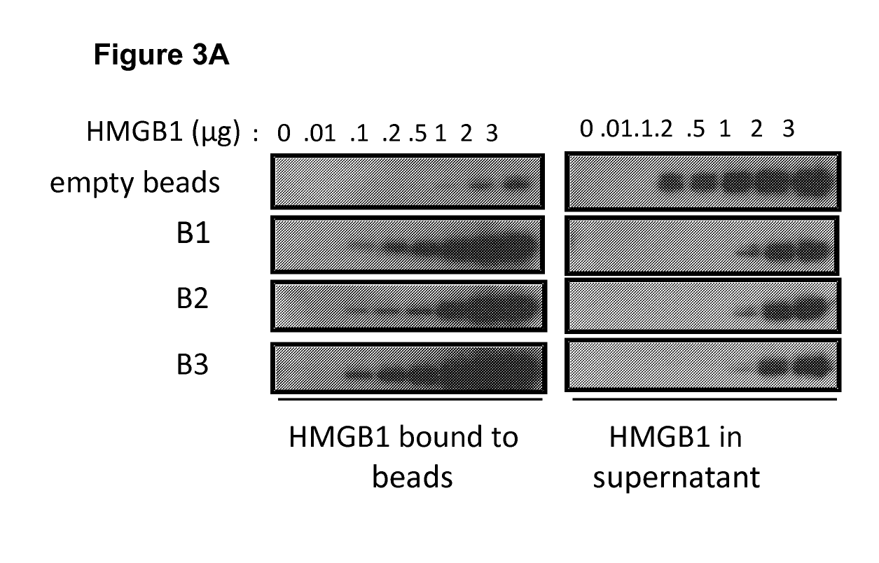

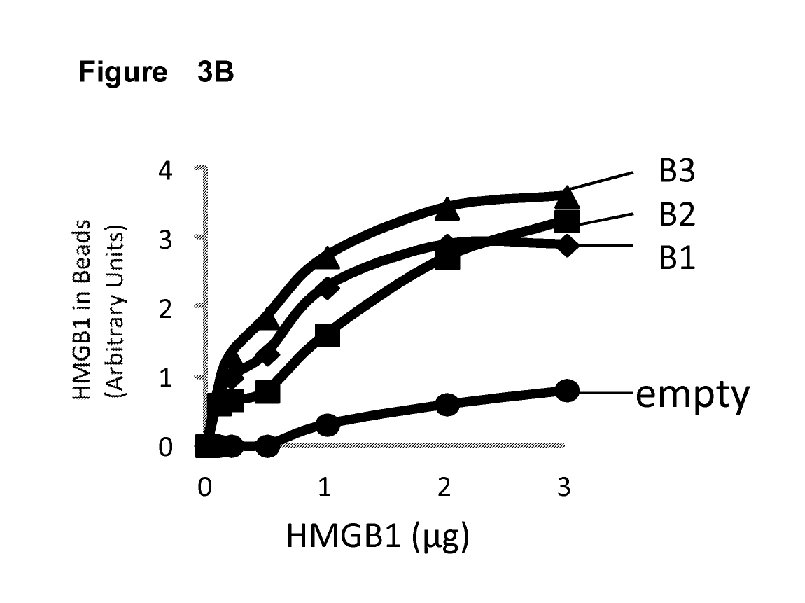

A binding-saturation approach was used to determine the maximal binding of DNA-beads to HMGB1. When constant amounts of DNA beads (20 μl) were incubated with increasing amounts of HMGB1, HMGB1 binds to the DNA beads in a concentration-dependent fashion, with the maximal binding for SEQ ID NO:1-beads, SEQ ID NO:2-beads, and 4 way junction-beads were 63 and 446 μg/ml drained beads, respectively. In comparison, there was no appreciable amount of HMGB1 binding in control beads lacking DNA (FIGS. 3A and 3B).

The binding ratio of HMGB1 to DNA beads was calculated based on the maximum binding of HMGB1 to a constant amount of DNA beads. HMGB1 binds to the DNA has a molar ratio of approximately 3:1 for S1 and S2 DNA, and a ratio of 6:1 for S3 DNA. Binding of 1 μg HMGB1 requires about 40 ng (for S1 and S2) and 280 ng for S3 DNA, respectively. DNA-beads were able to bind HMGB 1 with a capacity of 7.6 μM (every liter of DNA-beads binds to 7.6 micromolar of HMGB1), and each immobilized DNA molecule was able to bind three HMGB1 molecules (DNA:HMGB1 ratio=1:3).

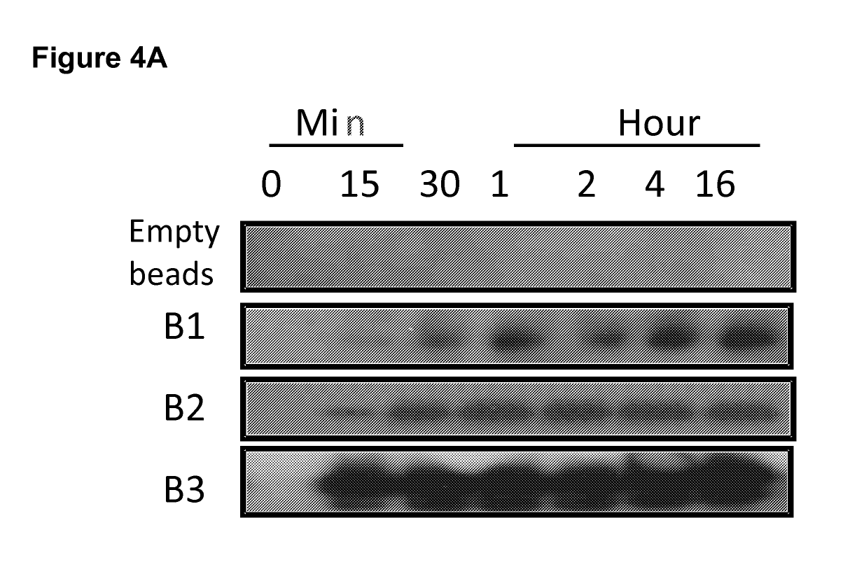

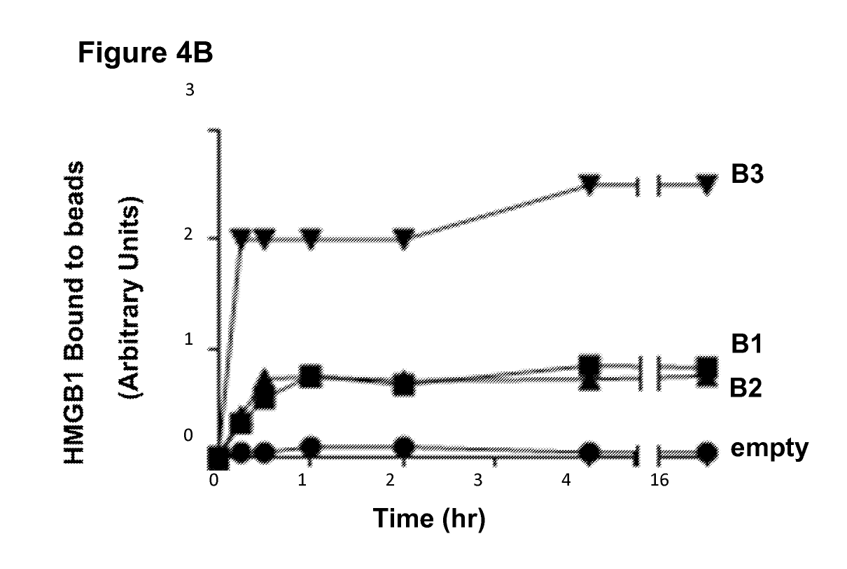

To determine the time kinetics of binding between beads and HMGB1, DNA-beads and HMGB1 binding was observed over time. DNA beads (20 μl) were incubated with HMGB1 (500 ng) for 0-4 hours at room temperature. HMGB1 captured on the beads was measured at each time point indicated (FIG. 4A). Both SEQ ID NO:1 and SEQ ID NO:2 on beads bind HMGB1 effectively and reach maximal binding at about 30 minutes after mixing together, whereas 4 way junction on beads binds to HMGB1 rapidly and reach saturation within 15 minutes (FIGS. 4A and 4B). Based on these findings, one hour of incubation was used for removing HMGB1 in subsequent in vitro experiments.

Binding affinity of DNA to HMGB1 (Biacore). Previous study has shown that 4 way junction binds HMGB1 with very high affinity (10-9 nM (11)). To definitively determine the binding affinity of DNA oligo SEQ ID NO:1 and SEQ ID NO:2 to HMGB1, biotinylated oligos and surface Plasmon resonance analysis (BIAcore) were used.

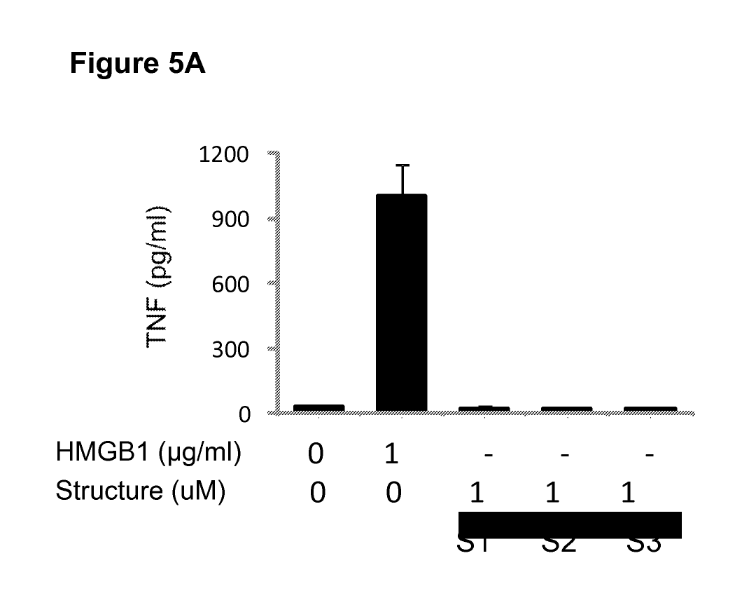

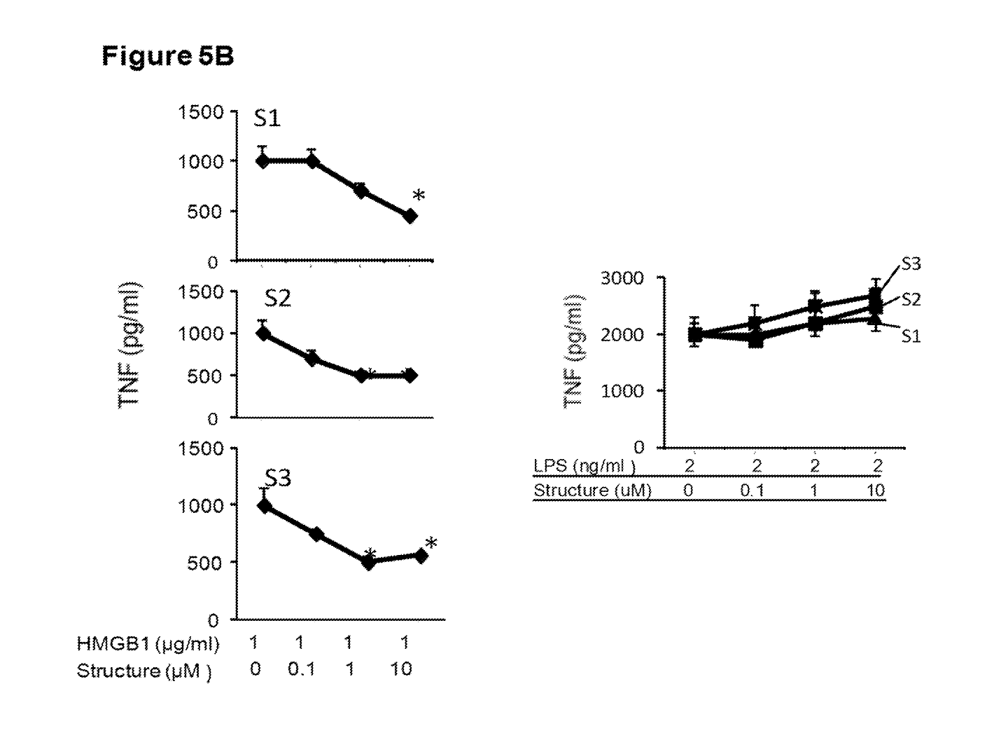

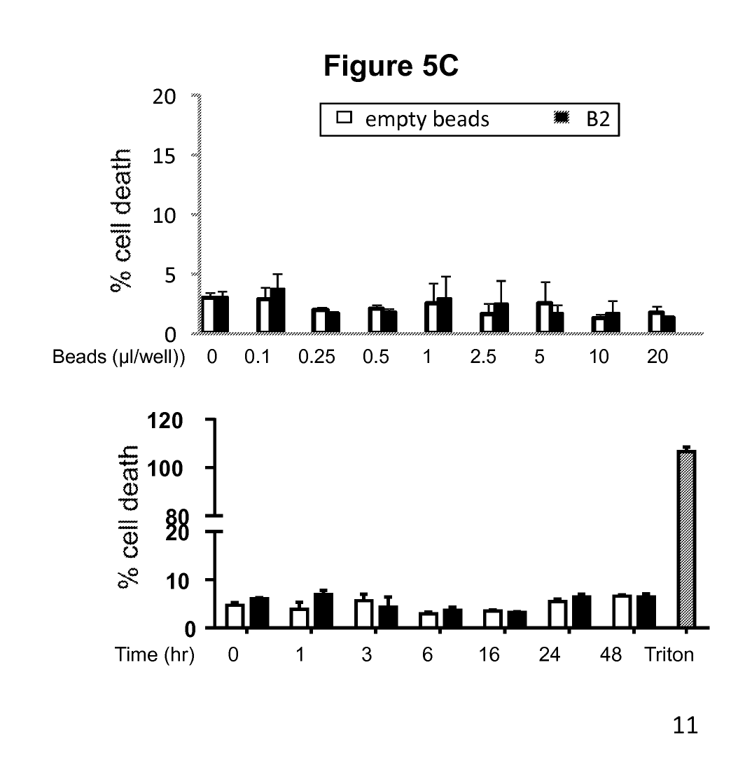

Suppressive effects of DNA oligos on HMGB1-induced TNF release on macrophages. It was examined whether these DNAs, which showed high binding affinity to HMGB1, are inert or could suppress HMGB1-mediated immune responses. As shown in FIG. 5A, human primary macrophages stimulated with HMGB1 had elevated TNF release, whereas none of the DNA had any significant effects in TNF stimulation at up to 1 μM concentrations. Moreover, addition of all DNA oligos had dose-dependent suppressive effects on HMGB1-induced TNF release from human macrophages (FIG. 5B). The suppressive effects of each DNA correlate with its binding affinity with HMGB1. Thus, all oligos used are inert and had inhibitory effects on HMGB1-induced TNF release. In contrast, addition of S1, S2 or S3 DNA did not appreciably alter LPS (2 ng/ml)-induced TNF release in macrophages, suggesting the effects of DNA is HMGB1-specific.

DNA beads are not cytotoxic. To examine whether DNA beads are toxic to cells, Caco-2 (human epithelial colorectal adenocarcinoma) cells were cultured with B2 or empty beads at increasing concentrations or for different time durations as indicated. Levels of secreted LDH were quantified in the cell supernatants as a measure of cell death. LDH levels were not significantly different between Caco-2 cells exposed to B2 or empty beads compared with medium alone. To further confirm whether exposure to beads induce cell death, LDH levels in the supernatant of Hela cells (human cervical cancer cell line) were also shown to be similar (FIG. 5C).

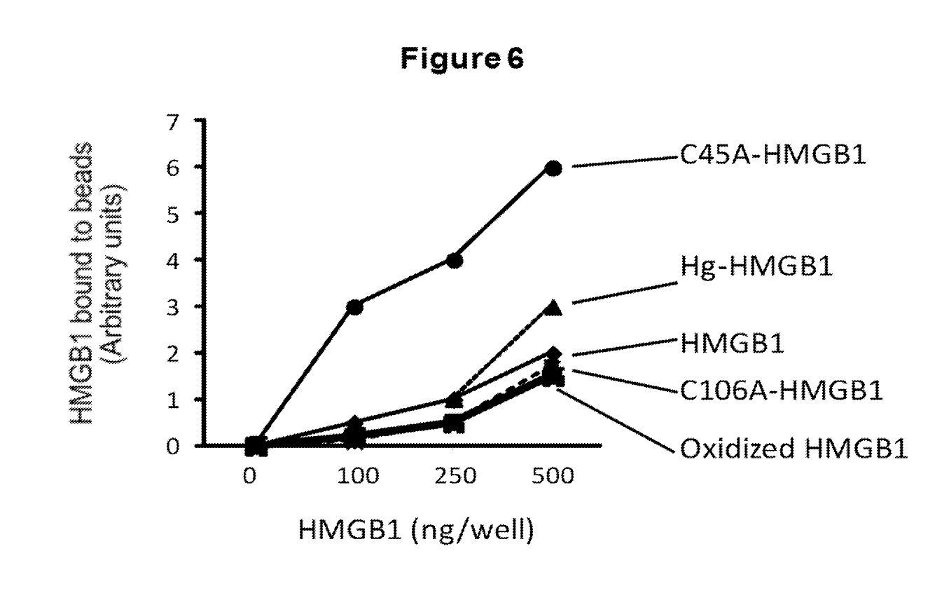

Binding of DNA beads to different forms of HMGB1. Previous studies showed that HMGB1 is present in different redox form in inflammatory diseases (23, 25). The binding of DNA oligos to different forms of HMGB1 was examined Increasing amounts of HMGB1 (100, 250 and 500 ng) were added to SEQ ID NO:1 beads (20 μl) and the mixture was incubated at room temperature for 2 hours. The mixture was then centrifuged, and HMGB1 bound to beads was revealed by Western blotting with anti-HMGB1 antibodies. SEQ ID NO:1 beads bind to all redox-modified HMGB1 proteins to a similar extent, with the exception that HMGB1 with cysteine at position 45 replaced by alanine (C45A) had even higher binding affinity (up to 5 fold) as compared to wild type (FIG. 6).

DNA beads do not bind to TNF. To evaluate the binding specificity of DNA beads to HMGB 1, increasing amounts of B2 beads were incubated with human TNF (200 ng) at room temperature for two hours and the amounts of free and bound TNF was analyzed. The DNA B2 beads did not sequester appreciable amounts of TNF.

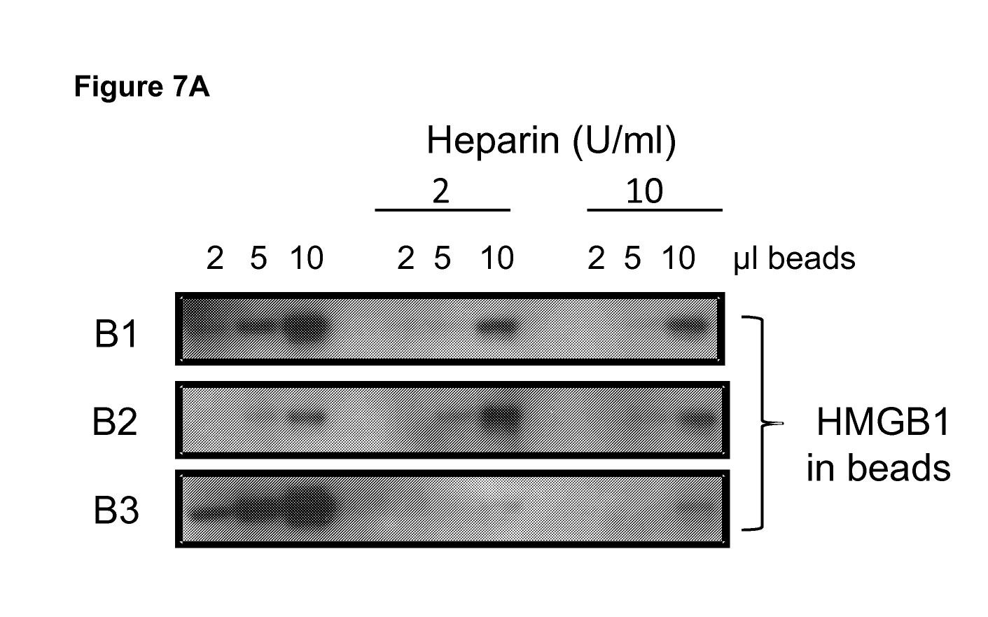

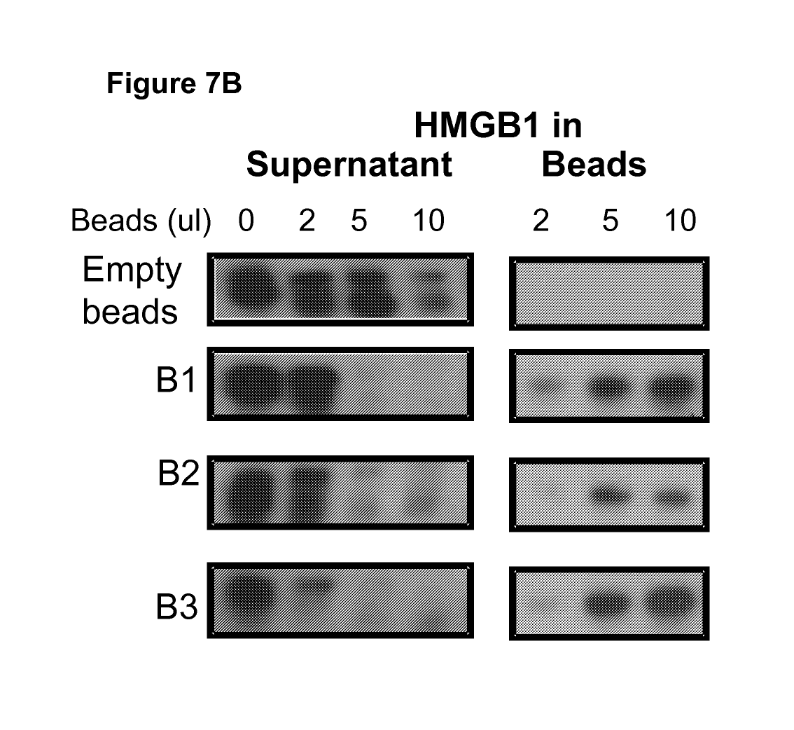

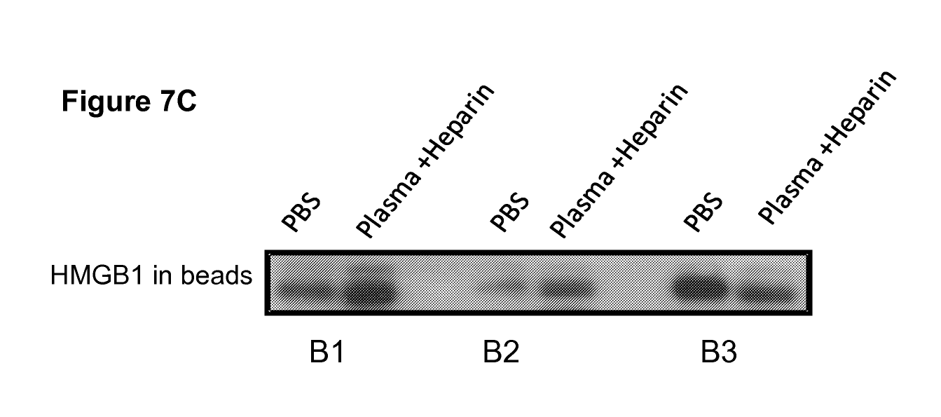

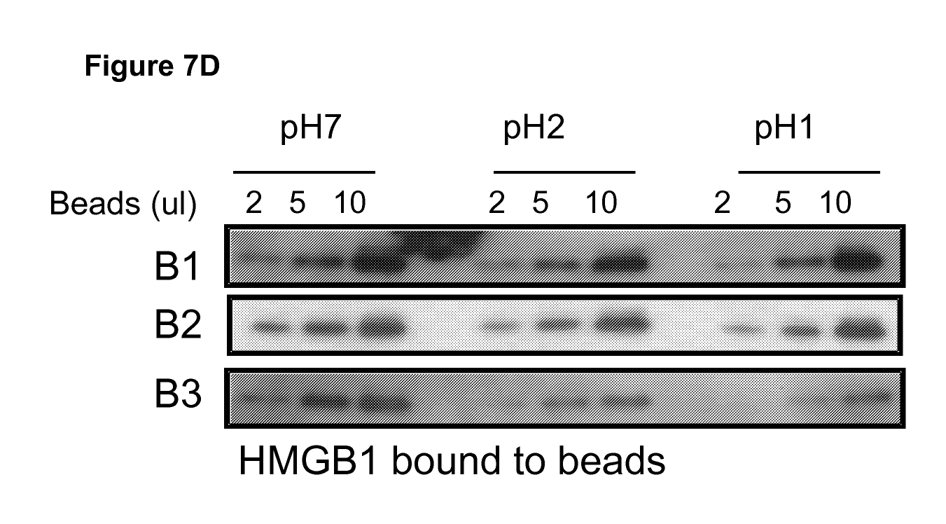

Binding of DNA beads to HMGB1 in the presence of heparin. With the aim to use these DNA-coated beads to remove HMGB1 in an extracorporeal device in sepsis model or in colitis patients, the functionality of these DNA-coated beads were examined in biological fluid and in cell assay systems. It was examined whether DNA beads can bind HMGB1 in the presence of acid, heparin or plasma, the environment that could be seen in a clinical scenario. HMGB1 (500 ng) was mixed with different amounts of DNA-coated beads (SEQ ID NO:1, SEQ ID NO:2 and 4 way junction) in the presence or absence of heparin (2 or 10 U/ml). SEQ ID NO:1 and SEQ ID NO:2 both bind HMGB1 in the presence of heparin, comparable as in the absence of heparin. The binding capacity of 4 way junction to HMGB1 was significantly diminished in the presence of heparin (up to 90%, FIG. 7A).

Binding of DNA beads to HMGB1 in the presence of plasma. Normal cow's plasma (20 μl) was incubated with HMGB1 for 1 hour at room temperature. After centrifugation, HMGB1 in the supernatant and beads was revealed by Western blot. Compared to beads only, DNA beads bind to HMGB1 similarly in the presence or absence of plasma; no significant differences were observed between SEQ ID NO:1, SEQ ID NO:2 or 4 way junction (FIGS. 7B and 7C). Thus, all 3 DNA beads can bind HMGB1 in the presence of plasma.

Binding of DNA beads to HMGB1 in the presence of acid. As DNA beads can be administered by oral route, it is important to study whether the beads are stable and able to bind HMGB1 after being exposed to acidic conditions of the stomach. This was examined by incubating DNA beads with HMGB1 at different pH (pH 1, 2 or 7) for an hour at room temperature. At the neutral pH, all three beads efficiently captured HMGB1 from solutions in a concentration-dependent manner, but only B1 and B2 retain the binding capacity at the low pH conditions (FIG. 7D).

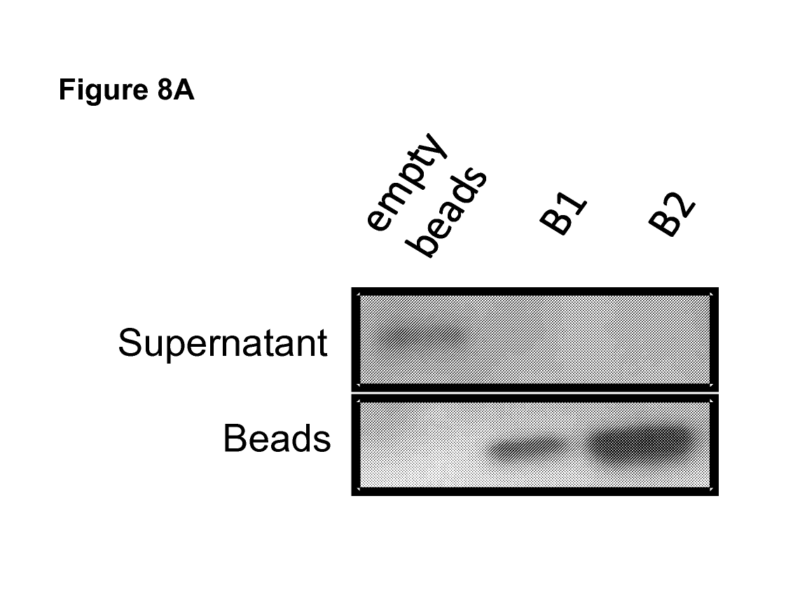

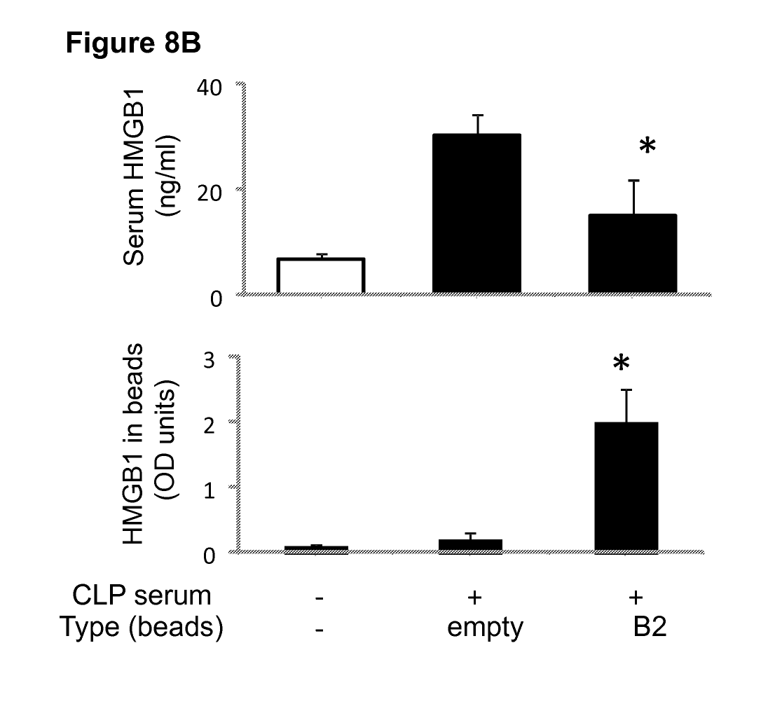

Ex vivo removal of HMGB1 from RAW 264.7 cell supernatant stimulated with LPS. When RAW 264.7 cells are stimulated with LPS, HMGB1 is known to be released into the supernatant. Having shown that DNA beads can bind HMGB1 in PBS, it was examined whether DNA beads can bind and remove HMGB1 in cell culture system. DNA-beads were incubated with LPS-stimulated RAW 264.7 cell supernatant. HMGB1 was measured both in the cell supernatant and in the DNA beads. As shown in FIG. 8A (upper), both SEQ ID NO:1- and SEQ ID NO:2-beads effectively bound and removed HMGB1 from RAW 264.7 cell supernatant as compared with control beads. As expected from this finding, HMGB1 is revealed from both SEQ ID NO:1 and SEQ ID NO:2 DNA beads whereas control beads (no DNA) did not show any appreciable amount of HMGB1 bound (FIG. 8A, lower). Furthermore, it seems that SEQ ID NO:2 captured more HMGB1 as compared to SEQ ID NO:1. In contrast, 4-way junction DNA-beads were not able to remove HMGB1 from RAW 264.7 cell supernatant (data not shown).

DNA beads bind and remove HMGB1 from mouse sepsis serum. Next, it was examined whether DNA-coated beads are able to remove HMGB1 from septic mice sera ex vivo. Septic mice following cecal ligation and puncture (CLP) had significantly elevated serum HMGB1 levels compared to normal controls (FIG. 8B, upper and (12)). SEQ ID NO:2 DNA-coated beads were added to these mice sera and incubated for 2 hours at 37° C. After centrifugation to separate beads and sera, HMGB1 levels in the septic sera were significantly reduced in SEQ ID NO:2-coated DNA beads as compared to beads lacking DNA (control) (FIG. 8B, upper). In agreement with these findings, after washing and elution from the beads, the recovered material showed HMGB1. Similarly, control beads (in the absence of DNA) did not bind HMGB1 whereas SEQ ID NO:2 DNA-coated beads did (FIG. 8B, lower). These data suggest that DNA-coated beads (for instance, contained in an extracorporeally located retention system through which the subject's blood is circulated) may be useful in removing HMGB1 from serum. In comparison, 4-way junction DNA-beads were not able to remove HMGB1 from mouse CLP serum (data not shown).

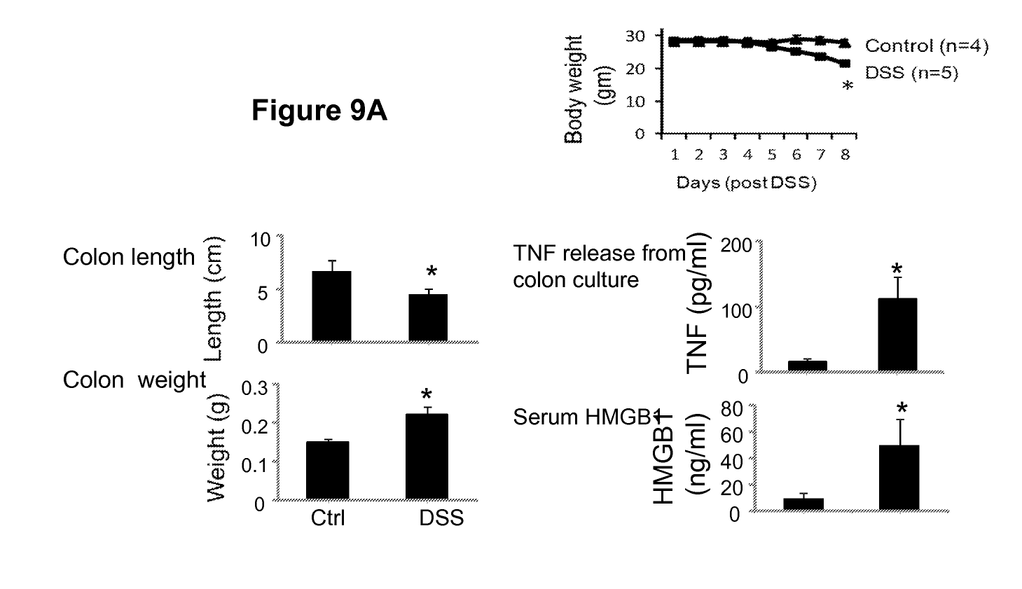

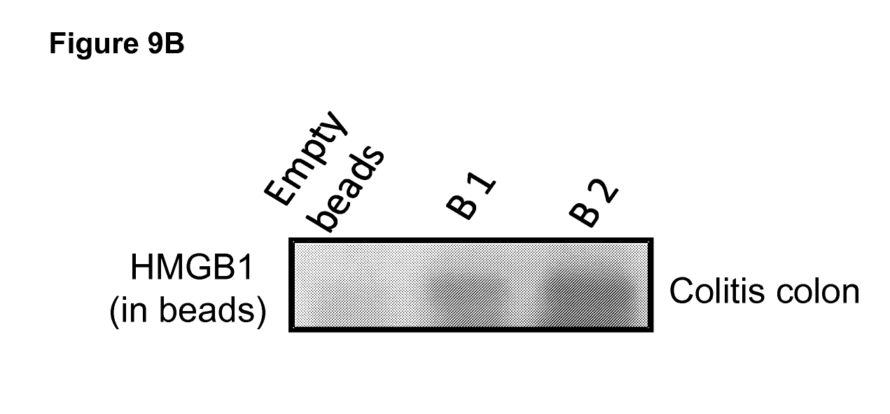

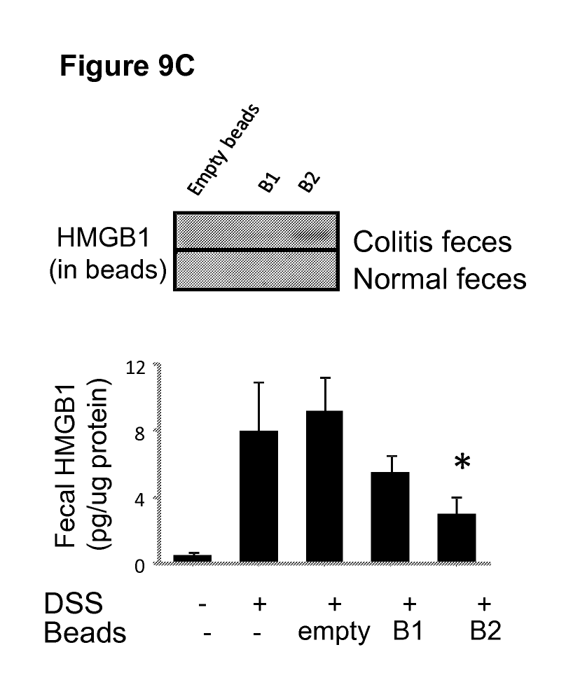

Ex vivo removal of HMGB1 from intestinal tissue and from stool of DSS induced colitis in mice. Estimation of the severity of colitis. BALB/c female mice (8-10 weeks of age) were subjected to 2% DSS water for 8 days. The severity of colitis in these mice was examined by daily observation of stool and measurement of body weight. The colitis was manifested by diarrhea, appearance of blood in the stool starting at day 4 after DSS water, and significant weight loss in the colitis group as compared to controls (FIG. 9A, left). At the end of 8 days with DSS water treatment, colitis mice had reduced colon length, increased colon weight and elevated serum HMGB1 levels compared to control mice (FIG. 9A, Right). Ex vivo culture of colon revealed that TNF release was not detectable in controls but increased from colon of colitis mice. Taken together, these findings clearly showed inflammation of this colitis model in mice.

Binding of HMGB1 from intestinal tissue and from stool of DSS induced colitis in mice. HMGB1 has been shown to be involved in the development of murine colitis and colitis-associated cancer (2). HMGB1 is abundantly found in stools of IBD patients and anti-HMGB1 treatment has been shown to be beneficial in this model (3). To evaluate if DNA beads can capture HMGB1 from colitis colon, SEQ ID NO:1 or SEQ ID NO:2-coated beads were administered to colons isolated from colitis or control mice. After interacting at 37° C. for 1 hour, HMGB1 content was measured in the recovered beads. Compared to empty beads, the addition of SEQ ID NO:1 or SEQ ID NO:2 beads to colitis colon led to the capture of significant amount of HMGB1 from colon culture ex vivo (FIG. 9B). Similarly, when fecal samples were incubated with SEQ ID NO:1 or SEQ ID NO:2-coated beads, both SEQ ID NO:1 and SEQ ID NO:2 coated beads captured HMGB1 from fecal matters of colitis mice as compared to beads lacking DNA (FIG. 9C). Hence, these findings indicate that HMGB1 is elevated both systemically and locally and contributes to the pathogenesis of colitis; and DNA beads, by binding and removing HMGB1, have potential therapeutic efficacy as a treatment approach in conditions such as colitis.

Covalently bound DNA are stable on beads and do not come off the beads. Fluorescent-labeled DNA coated beads were used to examine the stability of DNA-coated sepharose beads in biological fluid and temperature. The FAM-labeled DNA beads were incubated with fecal extract at 37° C. for two hours and the amounts of free DNA in the supernatant were evaluated. There were no considerable differences in the amount of fluorescent DNA released in the supernatant between beads exposed to fecal extract or not. The data also confirmed previous studies which showed that covalently bound antibodies to polystyrene latex beads are stable and that antibodies do not fall off beads (26).

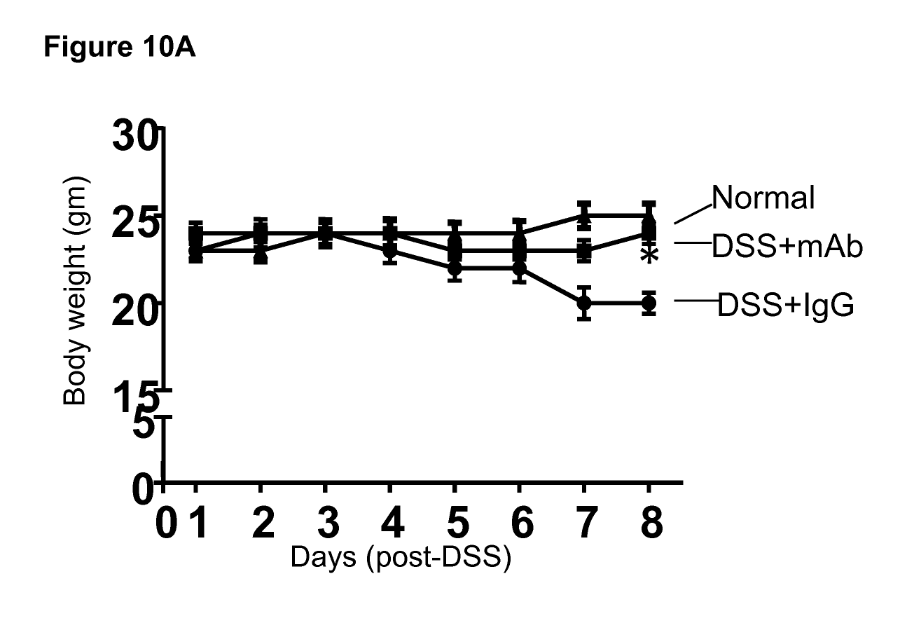

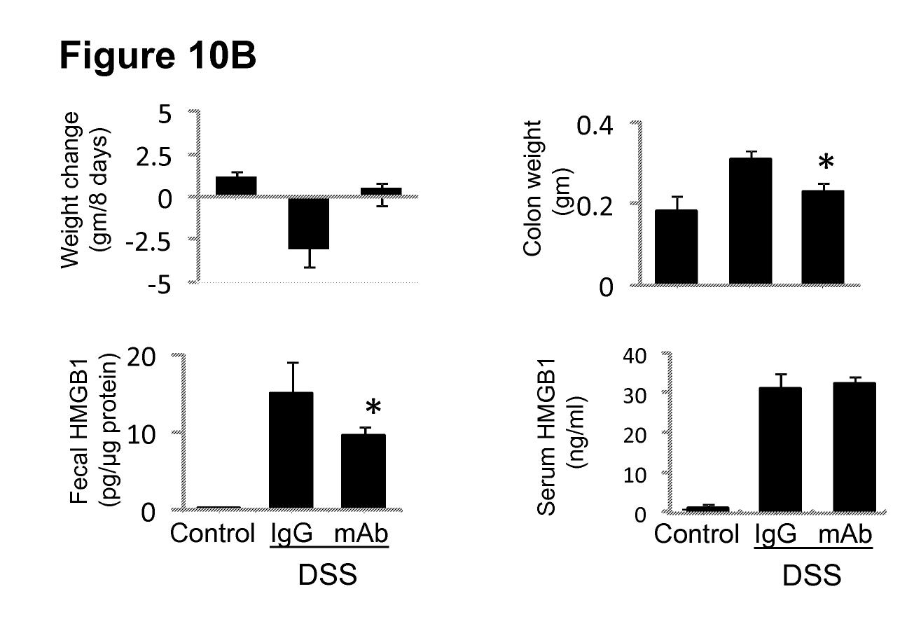

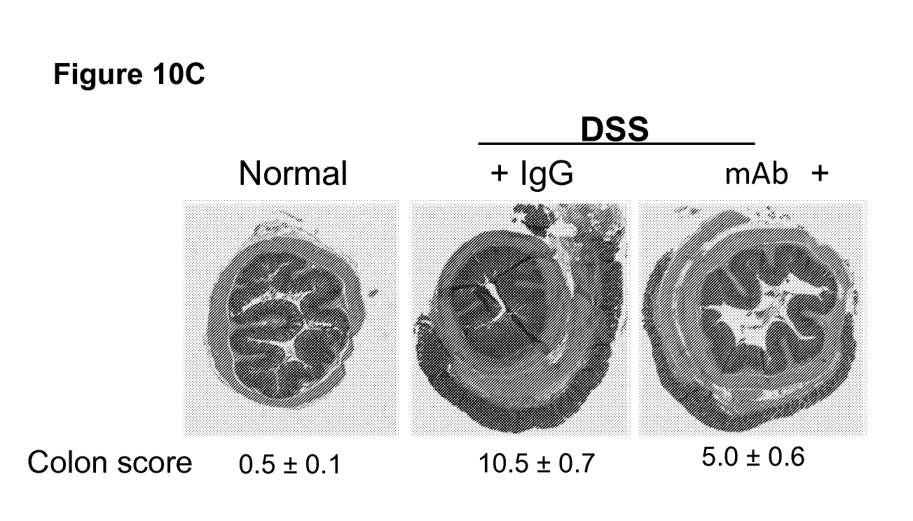

Administration of neutralizing anti-HMGB1 antibodies ameliorates DSS-induced colon injury and inflammation in mice. To determine whether neutralization of HMGB1 in colitis would improve disease outcome, female BALB/c mice were given 4% DSS in drinking water to induce colitis, and were treated with monoclonal anti-HMGB1 antibodies or control IgG at (10 μg/mouse on days 0, 1, 2, 4 and 6 after DSS administration). After 5 days of DSS administration, severe illness that was characterized by bloody diarrhea and severe wasting disease was observed. However, the relative body weight reduced at 8 days after DSS treatment in the IgG treated control group (−3.1 gm/8 days), whereas treatment of mice with neutralizing anti-HMGB1 antibody increased the body weight (0.5 gm/8 days, p<0.05 vs. empty beads, n=20 mice/group, FIG. 10A-B). These differences were directly reflected in the degree of injury as shown by formed fecal pellets, reduction in colonic wall thickening and fecal HMGB1 levels in antibody-treated groups when compared to IgG treated controls (FIGS. 10B and 10C). Treatment with anti-HMGB1 antibody did not significantly decrease serum HMGB1 levels, because neutralizing HMGB1 antibodies did not increase serum clearance of HMGB1, as observed previously (12). In agreement with these findings, histological evaluation of the colons revealed the colitis colon was characterized by loss of crypts, colon wall thickening and inflammatory cell infiltration compared to normal controls. This histological damage was significantly reduced and a doubling in histological scores was observed in HMGB1 antibody-treated group when compared to IgG controls (FIG. 10C).

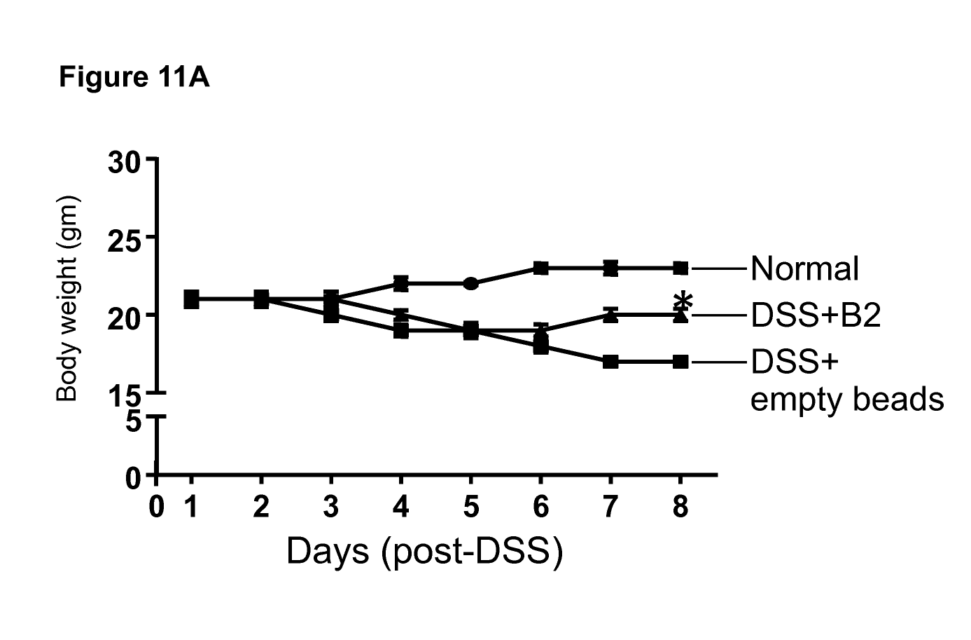

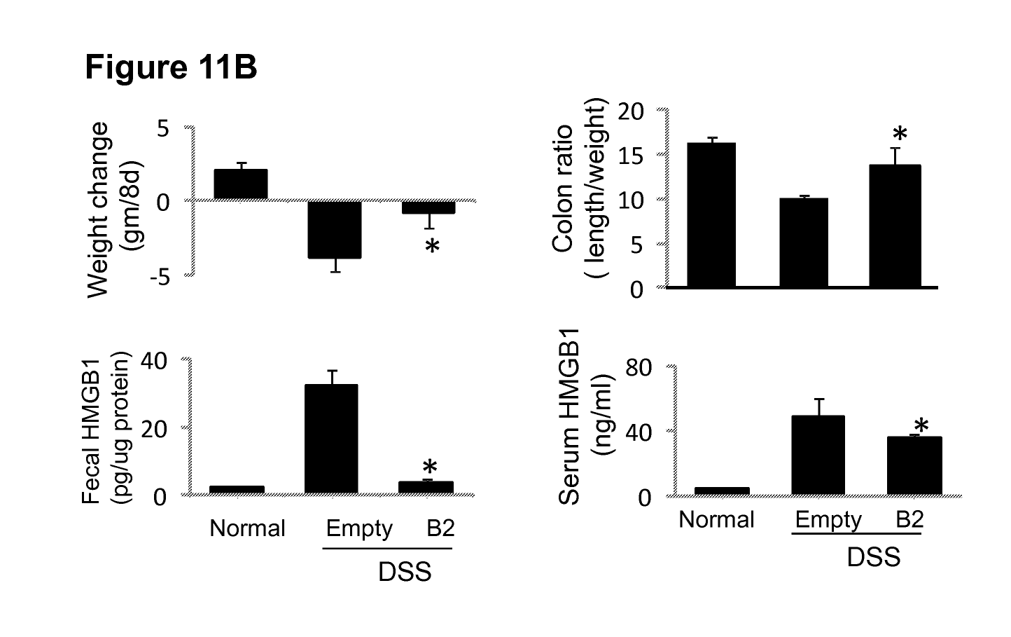

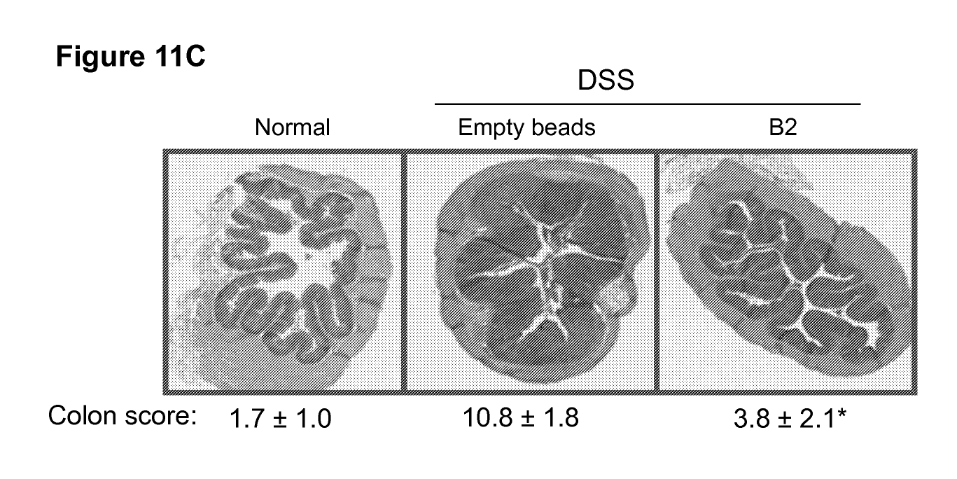

Administration of B2 beads ameliorates colitis-induced inflammation in both IL-10 KO and DSS-induced colitis in mice. To elucidate whether administration of HMGB1-specific DNA-coated beads can ameliorate inflammation through direct mucosal effects, the effects of DNA beads in modulating disease severity were examined in models of both DSS-induced colitis and IL-10 KO mice that spontaneously developed colitis (28). B2 or empty beads were administered per os. In DSS-induced colitis, treatment with B2 beads significantly increased body weight compared with empty beads treated mice (weight change=−0.8±0.4 gm/8 days in B2 group and −3.8±0.5 gm/8 days in empty beads treated groups respectively. N=10 mice per group, *P<0.05 vs. empty beads group, FIG. 11A); along with decreased colonic wall thickening as revealed by the ratio of colon length and weight and significant improved histological scores of the colon (scores=3.8±2.1 in B2 group vs. 10.8±1.8 in empty beads treated groups. P<0.05, FIG. 11B). Colitis-induced elevated HMGB1 levels in both serum and feces were reduced with treatment of B2 beads, along with improved colon scores shown by histological evaluation of the colons as compared to empty beads group (FIGS. 11B-C). Compared to normal mice, DSS induced colon damage including significant wall swelling, derangement and lesions of crypts, destruction of mucosa and sub-mucosa. Treatment with B2 beads had much improved histological changes as revealed by less tissue swelling, recovery of crypt structure as compared to empty beads-treated group (FIG. 11C).

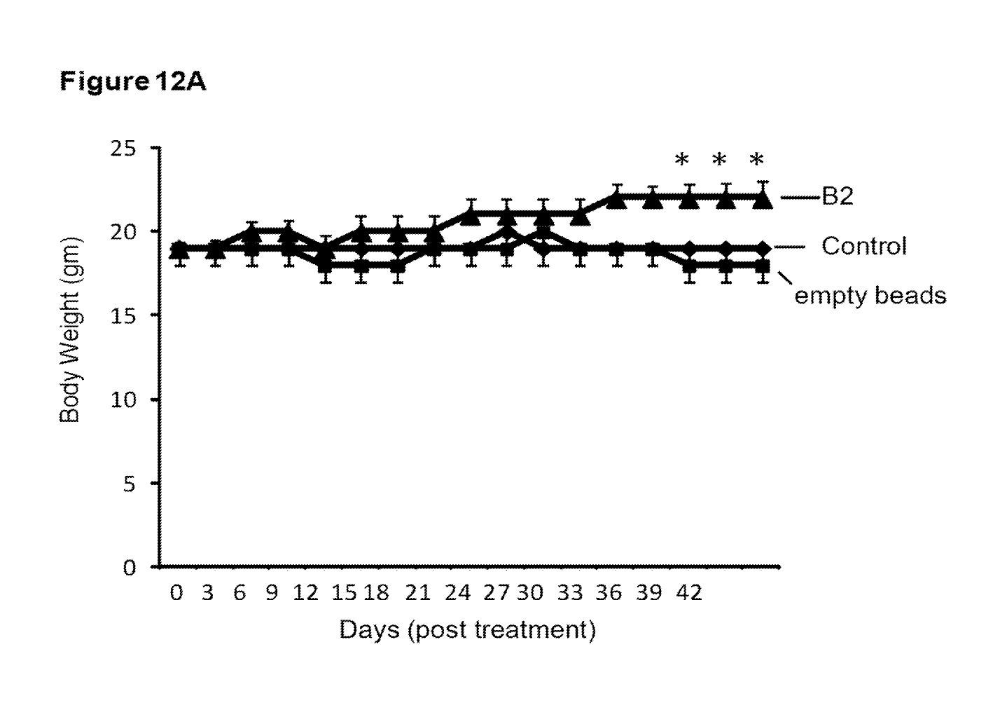

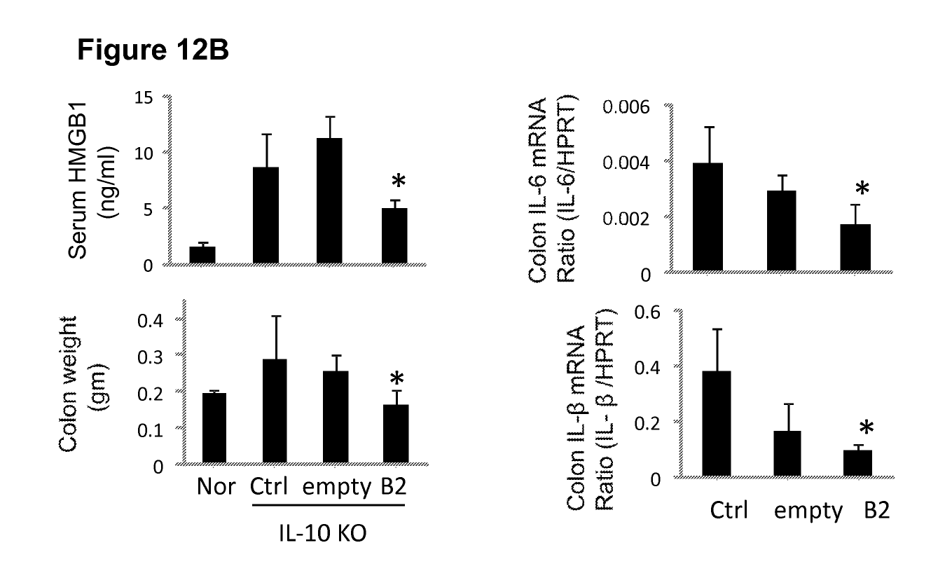

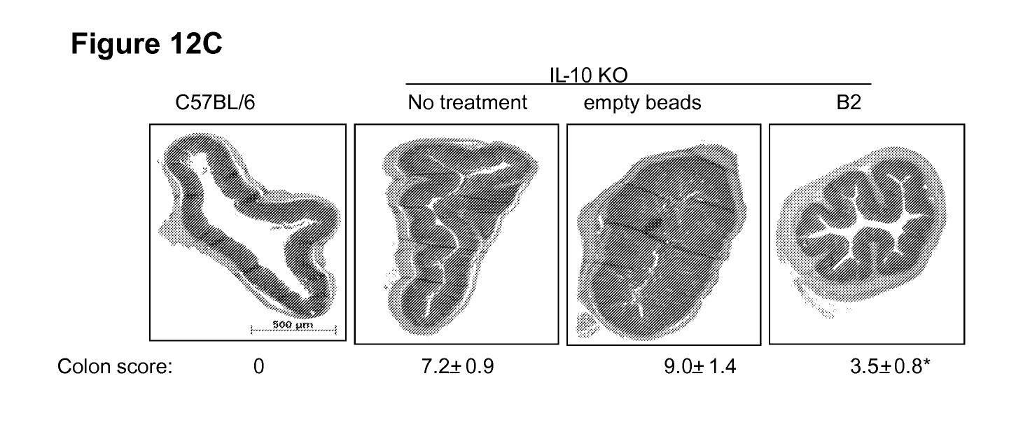

In agreement with these findings, in IL-10 KO mice, administration of B2 beads significantly increased body weight compared to empty beads or untreated (final body weight=23±2 in B2 group, 19±2 or 18±3 in untreated or empty beads treated groups respectively. N=5 or 7 mice per group, *P<0.05 B2 vs. empty beads group, FIG. 12A). Gross inspection of the colon revealed decreased colitis-induced colon weight in B2-treated group, as compared to mice treated with empty beads. Consistent with these findings, mice treated with B2 beads exhibited a significant reduction in serum HMGB1, mRNA expression of colon IL-6 and IL-1 beta when compared to the empty beads group (FIG. 12B). Histological evaluation demonstrated severe colon wall thickening in groups of untreated or empty beads mice, whereas significant improvement in B2-treated mice as revealed by the histological scores of the colons (score=3.5±0.8 in B2 group, and 7.2±0.9 or 9.0±1.4 in untreated or empty beads treated groups respectively. P<0.05, B2 vs. empty beads groups. FIG. 12C). Combined with the DSS-colitis model, these data showed that DNA beads are effective in reducing inflammation in two different models of colitis.

The present studies have established a novel approach using HMGB1-specific DNA-coated beads to effectively bind and sequester HMGB1 as demonstrated in clinically relevant models of colitis. Given the high affinity binding of DNA to HMGB1, this DNA-based HMGB1 sequester therapy in IBD provides several advantages. Firstly, the DNA beads are stable in acidic conditions as well as in fecal microenvironment. Covalently-conjugated beads are stable in acidic conditions and can be administered by oral route. Furthermore, the DNA beads are stable in the fecal microenvironment. However, even if the DNA beads lose some of the bound DNA, minimal toxicity would be expected since these DNA molecules are inert, not cytotoxic, and will be secreted with fecal matter. Secondly, since DNA beads can be administered directly to the gastrointestinal tract by oral route and hence enriched in the colon as compared to a regimen given systemically, systemic side effects such as generating anti-DNA antibodies or toxicity due to clearance should be avoided. Finally, DNA-based beads are non-toxic to the animals or cultured epithelial cells. Thus, the treatment strategy provided herein is feasible, effective, specific and safe, and opens a new avenue for blocking HMGB1 as a therapeutic approach in conditions such as IBD.

TABLE 1
Design of DNA oligos.

| DNA/structure | Oligos | Sequence 5′ to 3′ | Affinity (Kd) |
|---|---|---|---|
| 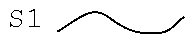 | 1 | 5′-AmC6AGCATGAGGTTCCTGATGCT | 5 nM |
| 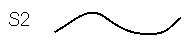 | 2 | AmC6TGGATGAGCTTCCTGATGTC | 5 nM |
| 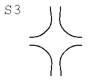 | 3 4 5 6 | AmC6CCCTATAACCCCTGCATTGAATTCCAGTCTGATAA GTAGTCGTGATAGGTGCAGGGGTTATAGGG AACAGTAGCTCTTATTCGAGCTCGCGCCCTATCACGACTA TTATCAGACTGGAATTCAAGCGCGAGCTCGAATAAGAGCTACTGT | 1 nM (6) |
| 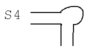 | 7 8 | AmC6CTTGCATTGAAATTTCTTTCC GAACGTAACAAAGAAAGG | 22 nM (7) |

SEQ ID NOs from top to bottom, respectively, SEQ ID NO: 1, 2, 5, 6, 7, 8, 3 and 4.

All oligonucleotides were custom made from Genemed Synthesis, Inc. with over 90% purity. An amino group linker has been conjugated to the 5′ of the oligos as indicated. In order to prevent DNase degradation, all oligos were synthesized with phosphorothioate (for SEQ ID NO:1 and SEQ ID NO:2) or phosphodiester backbone (for 4 way junction and duplex) throughout the sequences. The C6 amino linker is: NH₂(CH₂)₆O—P(O)₂—O-DNA. The DNA in between base (A,G,C,T) is base-O—P(═O)S—O-base.

### REFERENCES

1. Garland C F, Lilienfeld A M, Mendeloff A I, Markowitz J A, Terrell K B, Garland F C. Incidence rates of ulcerative colitis and Crohn's disease in fifteen areas of the United States. Gastroenterology. 1981; 81(6):1115-24.
2. Maeda S, Hikiba Y, Shibata W, Ohmae T, Yanai A, Ogura K, et al. Essential roles of high-mobility group box 1 in the development of murine colitis and colitis-associated cancer. Biochemical and biophysical research communications. 2007; 360(2):394-400.
3. Dave S H, Tilstra J S, Matsuoka K, Li F, DeMarco R A, Beer-Stolz D, et al. Ethyl pyruvate decreases HMGB1 release and ameliorates murine colitis. Journal of leukocyte biology. 2009; 86(3):633-43.
4. Vitali R, Stronati L, Negroni A, Di Nardo G, Pierdomenico M, del Giudice E, et al. Fecal HMGB1 is a novel marker of intestinal mucosal inflammation in pediatric inflammatory bowel disease. The American journal of gastroenterology. 2011; 106(11):2029-40.
5. Wirtz S, Neufert C, Weigmann B, Neurath M F. Chemically induced mouse models of intestinal inflammation. Nature protocols. 2007; 2(3):541-6.
6. Bianchi M E. Interaction of a protein from rat liver nuclei with cruciform DNA. The EMBO journal. 1988; 7(3):843-9.
7. Musumeci D, Bucci E M, Roviello G N, Sapio R, Valente M, Moccia M, et al. DNA-based strategies for blocking HMGB1 cytokine activity: design, synthesis and preliminary in vitro/in vivo assays of DNA and DNA-like duplexes. Molecular bioSystems. 2011; 7(5):1742-52.
8. Yanai H, Chiba S, Ban T, Nakaima Y, Onoe T, Honda K, et al. Suppression of immune responses by nonimmunogenic oligodeoxynucleotides with high affinity for high-mobility group box proteins (HMGBs). Proceedings of the National Academy of Sciences of the United States of America. 2011; 108(28):11542-7.
9. Chockalingam P S, Jurado L A, Jarrett H W. DNA affinity chromatography. Molecular biotechnology. 2001; 19(2):189-99.
10. Li J, Wang H, Mason J M, Levine J, Yu M, Ulloa L, Czura C J, Tracey K J and Yang H. 2004. Recombinant HMGB1 with cytokine-stimulating activity. J Immunol Methods. 289:211-223.
11. Hill, D A, Reeves R. Competition between HMG-I(Y), HMG-1 and histone H1 on four-way junction DNA. Nucleic Acids Res. 1997 17:3523-31.
12. Yang H, Ochani M, Li J H, Qiang X, Tanovic M, Harris H E, Susarla S M, Ulloa L, Wang H, DiRaimo R, Czura C J, Wang H C, Roth J, Warren H S, Fink M P, Fenton M J, Andersson U and Tracey K J. 2004. Reversing established sepsis with antagonists of endogenous HMGB1. Proc Natl Acad Sci. USA. 101:296-301.
13. Yang H, Hreggvidsdottir H S, Palmblad K, Wang H, Ochani M, Li J, Lu B, Chavan S, Rosas-Ballina M, Al-Abed Y, Akira S, Bierhaus A, Erlandsson-Harris H, Andersson U, Tracey K J. 2010. A critical cysteine is required for HMGB1 binding to Toll-like receptor 4 and activation of macrophage cytokine release. Proc Natl Acad Sci USA. 2010 Jun. 29; 107(26):11942-7.
14. Yang H, Tracey K J. 2010. Targeting HMGB1 in inflammation. Biochim Biophys Acta. 1799:149-156.
15. Yang H, Lundbäck P, Ottosson L, Erlandsson-Harris H, Bianchi M E, Venereau E, Al-Abed Y, Andersson U, Tracey K J, Antoine D J. 2012. Redox modification of cysteine residues regulates the cytokine activity of HMGB1. Mol Med. 18:250-259.
16. Qin S, Wang H, Yuan R, Li H, Ochani M, Ochani K, Rosas-Ballina M, Czura C J, Huston J M, Miller E, Lin X, Sherry B, Kumar A, LaRosa G, Newman W, Tracey K J, Yang H. 2006. Role of HMGB1 in apoptosis-mediated sepsis lethality. J Exp Med. 203:1637-1641.
17. Siiman Burshteyn A, Insausti M E. Covalently bound antibody on polystyrene latex beads: formation, stability, and use in analyses of white blood cell populations. J Colloid Interface Sci. 234: 44-58, 2001
18. Kovács T, Varga G, Erces D, T\acute{ó}kés T, Tiszlavicz L, Ghyczy M, Vécsei L, Boros M, Kaszaki J. [Comparative study of novel therapeutic possibilities in animal experimental model of inflammatory bowel disease] Magy Seb. 2012 August; 65(4):191-7. Hungarian.
19. Polanská E, Dob\check{s}áková Z, Dvo\check{r}á\check{c}ková M, Fajkus J, \check{S}tros M. HMGB1 gene knockout in mouse embryonic fibroblasts results in reduced telomerase activity and telomere dysfunction. Chromosoma. 2012 August; 121(4):419-31. Epub 2012 Apr. 28.
20. Yanai H, Ban T, Wang Z, Choi M K, Kawamura T, Negishi H, Nakasato M, Lu Y, Hangai S, Koshiba R, Savitsky D, Ronfani L, Akira S, Bianchi M E, Honda K, Tamura T, Kodama T, Taniguchi T. HMGB proteins function as universal sentinels for nucleic-acid-mediated innate immune responses. Nature. 2009 Nov. 5; 462(7269):99-103.
21. Wang H, Bloom O, Zhang M, Vishnubhakat J M, Ombrellino M, Che J, Frazier A, Yang H, Ivanova S, Borovikova L, Manogue K R, Faist E, Abraham E, Andersson J, Andersson U, Molina P E, Abumrad N N, Sama A, Tracey K J. HMG-1 as a late mediator of endotoxin lethality in mice. Science. 1999 Jul. 9; 285(5425):248-51.
22. Li J, Wang H, Mason J M, Levine J, Yu M, Ulloa L, Czura C J, Tracey K J, Yang H. Recombinant HMGB1 with cytokine-stimulating activity. J Immunol Methods. 2004 June; 289(1-2):211-23.
23. Yang H, Lundbäck P, Ottosson L, Erlandsson-Harris H, Venereau E, Bianchi M E, Al-Abed Y, Andersson U, Tracey K J, Antoine D J. Redox modification of cysteine residues regulates the cytokine activity of high mobility group box-1 (HMGB1). Mol Med. 2012 Mar. 30; 18:250-9.
24. Qin S, Wang H, Yuan R, Li H, Ochani M, Ochani K, Rosas-Ballina M, Czura C J, Huston J M, Miller E, Lin X, Sherry B, Kumar A, Larosa G, Newman W, Tracey K J, Yang H. Role of HMGB1 in apoptosis-mediated sepsis lethality. J Exp Med. 2006 Jul. 10; 203(7):1637-42. Epub 2006 Jul. 3.
25. Antoine D J, Williams D P, Kipar A, Laverty H, Park B K. Diet restriction inhibits apoptosis and HMGB1 oxidation and promotes inflammatory cell recruitment during acetaminophen hepatotoxicity. Mol Med. 2010 November-December; 16(11-12):479-90. Epub 2010 Aug. 27.
26. Siiman O, Burshteyn A, Insausti M E. Covalently Bound Antibody on Polystyrene Latex Beads: Formation, Stability, and Use in Analyses of White Blood Cell Populations. J Colloid Interface Sci. 2001 Feb. 1; 234(1):44-58.
27. Livak K J, Schmittgen T D (2001) Analysis of relative gene expression data using real-time quantitative PCR and the 2(-Delta Delta C(T)) Method. Methods 25: 402-408.
28. Kuhn R, Lohler J, Rennick D, Rajewsky K, Muller W (1993) Interleukin-10-deficient mice develop chronic enterocolitis. Cell 75: 263-274.

1. A method of treating a subject with a disease or condition selected from the group consisting of an inflammatory cascade, an inflammatory bowel disease, Crohn's disease, colitis, ulcerative colitis, colitis-associated cancer and a condition that would benefit from reducing the deleterious effects of high-mobility group box 1 (HMGB1), the method comprising administering beads coated with one or more agents that bind to HMGB1 to the gastrointestinal tract of the subject in an amount effective to treat the disease or condition.

2. A method of reducing the level of HMGB1 in the gastrointestinal tract of a subject comprising administering beads coated with one or more agents that bind to HMGB1 to the gastrointestinal tract of the subject in an amount effective to reduce the level of HMGB1 in the gastrointestinal tract of a subject.

3. (canceled)

4. The method of claim 1, wherein the beads are administered using a tube or an endoscope.

5-7. (canceled)

8. The method of claim 1, wherein the beads have an enteric coating.

9. The method of claim 1, wherein the beads not absorbable by the gastrointestinal tract.

10. (canceled)

11. The method of claim 1, wherein the agent comprises an antibody, an antibody fragment, a peptide, a synthetic compound, a peptide nucleic acid and/or a nucleic acid that binds HMGB1.

12. The method of claim 1, wherein the agent comprises a nucleic acid.

13. The method of claim 12, wherein the nucleic acid is one or more of a single linear chain, a duplex of two chains, a 4-way junction of four chains, a cisplatin-modified nucleic acid, a kinked nucleic acid, a hemi-catenated nucleic acid, or a nucleic acid containing a loop.

14. The method of claim 12, wherein the nucleic acid is a single linear chain consisting of 15-30 nucleotides.

15-16. (canceled)

17. The method of claim 14, wherein the nucleic acid comprises a sequence that is at least 80% identical to the sequence X₁GX₂ATGAGX₃TTCCTGATGCT (SEQ ID NO:9), where X₁ and X₂ are independently A, C, G or T, and X₃ is C or G.

18-21. (canceled)

22. The method of claim 14, wherein the nucleic acid comprises a sequence that is at least 80% identical to the sequence AGCATGAGGTTCCTGATGCT (SEQ ID NO:1).

23-26. (canceled)

27. The method of claim 14, wherein the nucleic acid comprises a sequence that is at least 80% identical to the sequence TGGATGAGCTTCCTGATGCT (SEQ 607826.1 ID NO:2).

28-31. (canceled)

32. The method of claim 12, wherein the nucleic acid comprises a 4-way junction of four chains and wherein each chain comprises a sequence that is at least 80% identical to the sequence set forth in SEQ ID NO:5, SEQ ID NO:6, SEQ ID NO:7, or SEQ ID NO:8.

33-34. (canceled)

35. The method of claim 12, wherein the nucleic acid comprises a duplex of two chains and wherein each chain comprises a sequence that is at least 80% identical to the sequence set forth in SEQ ID NO:3 or SEQ ID NO:4.

36-37. (canceled)

38. The method of claim 11, wherein the nucleic acid has a phosphorothioate backbone or phosphodiester backbone.

39. (canceled)

40. The method of claim 11, wherein the nucleic acid has a backbone between nucleotide bases of —O—P(═O)S—O—, where O— is the point of attachment to a base.

41. The method of claim 11, wherein the nucleic acid is attached to the beads using a carbon amino linker.

42. The method of claim 41, wherein the linker to the nucleic acid comprises NH₂(CH₂)₆O—PO₂—O-DNA, where DNA represents the nucleic acid.

43-44. (canceled)

45. A therapeutic bead for treating an inflammatory cascade, an inflammatory bowel disease, Crohn's disease, colitis, ulcerative colitis, colitis-associated cancer or a condition that would benefit from reducing the deleterious effects of HMGB1 comprising beads coated with one or more agents that bind to HMGB1.

46-84. (canceled)

85. The method of claim 1, wherein administration of beads coated with an agent that binds to HMGB1 reduces the level of HMGB1 in the gastrointestinal tract and stool.

86. (canceled)


SEQUENCE LISTING (15 sequences)

SEQ ID NO: 1 - DNA, 20 bp, Artificial Sequence
  binding sequence for HMGB1
agcatgaggttcctgatgct

SEQ ID NO: 2 - DNA, 20 bp, Artificial Sequence
  binding sequence for HMGB1
tggatgagcttcctgatgtc

SEQ ID NO: 3 - DNA, 21 bp, Artificial Sequence
  binding sequence for HMGB1
cttgcattgaaatttctttcc

SEQ ID NO: 4 - DNA, 18 bp, Artificial Sequence
  binding sequence for HMGB1
gaacgtaacaaagaaagg

SEQ ID NO: 5 - DNA, 35 bp, Artificial Sequence
  binding sequence for HMGB1
ccctataacccctgcattgaattccagtctgataa

SEQ ID NO: 6 - DNA, 30 bp, Artificial Sequence
  binding sequence for HMGB1
gtagtcgtgataggtgcaggggttataggg

SEQ ID NO: 7 - DNA, 40 bp, Artificial Sequence
  binding sequence for HMGB1
aacagtagctcttattcgagctcgcgccctatcacgacta

SEQ ID NO: 8 - DNA, 46 bp, Artificial Sequence
  binding sequence for HMGB1
tttatcagactggaattcaagcgcgagctcgaataagagctactgt

SEQ ID NO: 9 - DNA, 20 bp, Artificial Sequence
  binding sequence for HMGB1
ngnatgagnttcctgatgct

SEQ ID NO: 10 - protein, 215 aa, Homo sapiens [sequence omitted]

SEQ ID NO: 11 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 12 - DNA, 25 bp, Artificial Sequence
  forward primer for IL-6
gctaccaaactggatataatcagga

SEQ ID NO: 13 - DNA, 23 bp, Artificial Sequence
  reverse primer for IL-6
caggtagctatggtactccagaa

SEQ ID NO: 14 - DNA, 19 bp, Artificial Sequence
  forward primer for IL-1beta
agttgacggaccccaaaag

SEQ ID NO: 15 - DNA, 20 bp, Artificial Sequence
  reverse primer for IL-1beta
agctggatgctctcatcagg